(19) United States
(12) Patent Application Publication
(10) Pub. No.: US 2026/0002930 A1
(43) Pub. Date: Jan. 1, 2026

(54) BIOMARKERS FOR ALLERGEN IMMUNOTHERAPY

(71) Applicants: Monash University, Clayton (AU); Alfred Health, Melbourne (AU)

(72) Inventors: Menno C. van Zelm, Clayton (AU); Robyn Elizabeth O'Hehir, Clayton (AU); Craig I. McKenzie, Clayton (AU)

(21) Appl. No.: 18/868,547

(22) PCT Filed: May 24, 2023
(86) PCT No.: PCT/AU2023/050439

§ 371 Date: Nov. 22, 2024

(30) Foreign Application Priority Data
May 24, 2022 (AU) 2022901401

(51) Int. Cl. G01N 33/50; C12Q 1/6883; G01N 33/68

(52) U.S. Cl. CPC G01N 33/5052; C12Q 1/6883; G01N 33/5091; G01N 33/6869; C12Q 2600/106; C12Q 2600/158; G01N 2333/5406; G01N 2333/7055; G01N 2333/70596; G01N 2333/7155; G01N 2800/24; G01N 2800/52

(57) ABSTRACT

The present invention relates to the field of medical diagnostics. In particular, it relates to methods and kits for determining treatment efficacy of allergen immunotherapy, and for measuring or detecting biomarkers in a subject undergoing allergen immunotherapy. For example, the invention provides a method of determining efficacy of an allergen immunotherapy in a subject, the method comprising: providing a first sample obtained from a subject before receiving allergen immunotherapy; providing a second sample obtained from the subject who has received, or who is receiving, allergen immunotherapy; wherein the first and second samples comprise B-cells; and determining the level or amount of one or more biomarkers in B-cells, preferably allergen-specific B-cells, in the first and second samples, wherein the biomarkers are selected from the group consisting of IgE, CD29, CD69, IL13Rα, CD99, IgD, CXCR4, FCRL3, FCRL2, FCRL5, SIGLEC10, CDIc, CD23, and IL4Rα; wherein an increase in the level or amount of one or more biomarkers selected from IgE, CD29, IL13Rα, CD99, FCRL3, FCRL2, SIGLEC10, CD1c, CD23, and IL4Rα in B-cells in the second sample compared to the first sample indicates efficacy of an allergen immunotherapy in a subject, and/or wherein a decrease in the level or amount of one or more biomarkers selected from CD69, IgD, CXCR4, FCRL3, FCRL2, FCRL5, CD1c, CD23, IL4Rα in B-cells in the second sample compared to the first sample indicates efficacy of an allergen immunotherapy in a subject.

### FIELD OF THE INVENTION

The present invention relates to the field of medical diagnostics, prognosis and monitoring treatment efficacy. In particular, it relates to methods and kits for determining treatment efficacy of allergen immunotherapy, and for measuring or detecting biomarkers in a subject undergoing allergen immunotherapy.

### CROSS REFERENCE TO EARLIER APPLICATION

This application claims priority from Australian provisional application 2022901401, the entire contents of which are hereby incorporated by reference.

### BACKGROUND OF THE INVENTION

Allergic diseases are amongst the most common chronic immune-mediated disorders, and can manifest with enormous diversity in clinical severity and range of symptoms. As a result, there have been, and still are, major challenges in diagnosis, prediction of disease progression/evolution and treatment.

Traditionally, patients have been treated with one of two approaches: (a) symptom control by nonspecific pharmacotherapy through neutralization of effector molecules, for example anti-histamines; and (b) reprogramming of the immune response through specific allergen immunotherapy (AIT).

AIT through long term repeated exposure to low-doses of allergen has been applied for over a century making it the oldest form of immunotherapy, i.e. redirecting an existing immune response. However, as patients need to be treated for at least several years for AIT to be effective, and this can be accompanied by adverse reactions, treatment adherence is typically poor.

Over the past two decades, treatment of allergic diseases has dramatically changed with the introduction of biologicals. Specifically, monoclonal antibodies (mAb) against IgE, type 2 cytokines and their receptors, and small molecule inhibitors of signal transduction pathways have shown promise in modulating especially severe responses. However, these therapeutics are not curative, expensive, not effective in all patients, and regimens are often very long term to lifelong.

Pollen (grass, tree or weed) allergy is the leading cause of seasonal allergic rhinitis and/or asthma and house dust mite allergy is the leading cause of perennial allergic rhinitis and/or asthma globally. One particular allergic disease, pollen allergy, is a leading cause of seasonal allergic rhinitis worldwide, with 10-30% of the global population exhibiting sensitization to one or more grass pollen allergens. Allergies to grass pollen are a considerable burden on quality of life due to ongoing symptoms of rhinoconjunctivitis throughout the pollen season, anxiety in grass-covered areas such as public parks, the development of co-morbidities such as asthma and the financial costs of treatment and time off work or school. Furthermore, grass pollen allergy is the underlying cause of thunderstorm asthma which can overwhelm healthcare institutions from large numbers of patients suffering from acute asthmatic exacerbations that can be fatal in extreme cases. For example, during the November 2016 thunderstorm event in Melbourne, Australia there were more than 3,000 hospitalizations and 10 deaths.

With the challenges of patient commitment to long-term therapy with potentially high costs, there is a great need for objective biomarkers to predict treatment response, disease progression and/or evolution in a patient.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

In one aspect, the present invention provides a method of determining efficacy of an allergen immunotherapy in a subject, the method comprising:
providing a first sample obtained from a subject before receiving allergen immunotherapy;
providing a second sample obtained from the subject who has received, or who is receiving, allergen immunotherapy;
wherein the first and second samples comprise B-cells; and
determining the level or amount of one or more biomarkers in B-cells in the first and second samples, wherein the biomarkers are selected from the group consisting of IgE, CD29, CD69, IL13Rα, CD99, IgD, CXCR4, FCRL3, FCRL2, FCRL5, SIGLEC10, CD1c, CD23, and IL4Rα;
wherein an increase in the level or amount of one or more biomarkers selected from IgE, CD29, IL13Rα, CD99, FCRL3, FCRL2, SIGLEC10, CD1c, CD23, and IL4Rα in B-cells in the second sample compared to the first sample indicates efficacy of an allergen immunotherapy in a subject, and/or
wherein a decrease in the level or amount of one or more biomarkers selected from CD69, IgD, CXCR4, FCRL3, FCRL2, FCRL5, CD1c, CD23, and IL4Rα in B-cells in the second sample compared to the first sample indicates efficacy of an allergen immunotherapy in a subject.

In one embodiment of this aspect of the present invention, there may be at least a 1.5 fold increase in the level or amount of one or more biomarkers selected from IgE, CD29, IL13Rα, CD99, SIGLEC10, CD1c and CD23 in B-cells in a second sample compared to a first sample, which indicates efficacy of an allergen immunotherapy in a subject.

In one embodiment of this aspect of the present invention, there may be at least a 2-fold increase in the level or amount of one or more biomarkers, this may include any one of more of IgE, CD29, IL13Rα, CD99, SIGLEC10, and CD1c in B-cells in a second sample compared to a first sample, which indicates efficacy of an allergen immunotherapy in a subject.

In one embodiment of this aspect of the present invention, there may be at least a 2.5-fold increase in the level or amount of one or more biomarkers, this may include any one of more of IgE, CD29, IL13Rα and CD99 in B-cells in a second sample compared to a first sample, which indicates efficacy of an allergen immunotherapy in a subject.

In one embodiment of this aspect of the present invention, there may be at least a 3-fold increase in the level or amount of one or more biomarkers, this may include any one of more of IgE, CD29, and IL13Rα in B-cells in a second sample compared to a first sample, which indicates efficacy of an allergen immunotherapy in a subject.

In one embodiment of this aspect of the present invention, there may be at least a 4-fold increase in the level or amount of a biomarker, this may include CD29 in B-cells in a second sample compared to a first sample, which indicates efficacy of an allergen immunotherapy in a subject.

In one embodiment of this aspect of the present invention, there may be at least a 1.5 fold decrease in the level or amount of one or more biomarkers selected from CD69, IgD, CXCR4, FCRL3, FCRL2, FCRL5, CD1c, CD23 and IL4Rα in B-cells in a second sample compared to a first sample, which indicates efficacy of an allergen immunotherapy in a subject.

In one embodiment of this aspect of the present invention, there may be at least a 2-fold decrease in the level or amount of one or more biomarkers, this may include any one of more of CD69, IgD, CXCR4, FCRL3, FCRL2, FCRL5, CD23 and IL4Rα in B-cells in a second sample compared to a first sample, which indicates efficacy of an allergen immunotherapy in a subject.

In one embodiment of this aspect of the present invention, there may be at least a 2.5-fold decrease in the level or amount of one or more biomarkers, this may include any one of more of CD69, IgD, CXCR4 and FCRL3 in B-cells in a second sample compared to a first sample, which indicates efficacy of an allergen immunotherapy in a subject.

In one embodiment of this aspect of the present invention, there may be at least a 3-fold decrease in the level or amount of one or more biomarkers, this may include any one of more of IgD and CXCR4 in B-cells in a second sample compared to a first sample, which indicates efficacy of an allergen immunotherapy in a subject.

In one embodiment of this aspect of the present invention, there may be at least a 4-fold decrease in the level or amount of one or more biomarkers, this may include any one of more of IgD and CXCR4 in B-cells in a second sample compared to a first sample, which indicates efficacy of an allergen immunotherapy in a subject.

In another aspect, the present invention provides a method of determining efficacy of an allergen immunotherapy in a subject, the method comprising:
providing a first sample obtained from a subject before receiving allergen immunotherapy;
providing a second sample obtained from the subject who has received, or who is receiving, allergen immunotherapy;
wherein the first and second samples comprise B-cells; and
determining the level or amount of one or more biomarkers in B-cells in the first and second samples, wherein the biomarkers are IgG4, CD29, CD23 and IL4Rα;
wherein an increase in the level or amount of any one, two, three or all of IgG4, CD29, CD23 and IL4Rα, in B-cells in the second sample compared to the first sample indicates efficacy of an allergen immunotherapy in a subject. Preferably, the B-cells are memory B cells (Bmem), more preferably the Bmem are allergen specific Bmem, where the allergen is the same allergen provided in the allergen immunotherapy.

In this aspect, the method further comprises determining the level or amount of one or more additional biomarkers. The additional markers may be biomarkers that are described in Table 2 and/or Table 3. Preferably, the biomarkers are those that increase or decrease 1.5 fold, 2 fold, 3 fold or 4 fold as described in Table 2 and/or Table 3.

In another aspect, the present invention provides a method of determining efficacy of an allergen immunotherapy in a subject, the method comprising:
providing a first sample obtained from a subject before receiving allergen immunotherapy;
providing a second sample obtained from the subject who has received, or who is receiving, allergen immunotherapy;
wherein the first and second samples comprise B cells; and
determining the proportion or number of IgG+ Bmem or IgG4+ allergen specific Bmem in the first and second samples;
wherein an increase in the proportion or number of IgG+ Bmem or IgG4+ allergen specific Bmem in the second sample compared to the first sample indicates efficacy of an allergen immunotherapy in a subject.

In this aspect, the method further comprises determining the proportion or number of IgG+ Bmem or IgG4+ allergen specific Bmem expressing one or more additional biomarkers. The additional markers may be biomarkers that are described in Table 2 and/or Table 3. Preferably, the biomarkers are those that increase or decrease 1.5 fold, 2 fold, 3 fold or 4 fold as described in Table 2 and/or Table 3.

In another aspect, the present invention provides a method of determining efficacy of an allergen immunotherapy in a subject, the method comprising:
providing a first sample obtained from a subject before receiving allergen immunotherapy;
providing a second sample obtained from the subject who has received, or who is receiving, allergen immunotherapy;
wherein the first and second samples comprise B cells; and
determining the proportion or number of CD29+ allergen specific Bmem or CD29hi allergen specific Bmem in the first and second samples;
wherein an increase in the proportion or number of CD29+ allergen specific Bmem or CD29hi allergen specific Bmem in the second sample compared to the first sample indicates efficacy of an allergen immunotherapy in a subject.

In this aspect, the method further comprises determining the proportion or number of CD29+ allergen specific Bmem or CD29hi allergen specific Bmem expressing one or more additional biomarkers. The additional markers may be biomarkers that are described in Table 2 and/or Table 3. Preferably, the biomarkers are those that increase or decrease 1.5 fold, 2 fold, 3 fold or 4 fold as described in Table 2 and/or Table 3.

In another aspect, the present invention provides a method of determining efficacy of an allergen immunotherapy in a subject, the method comprising:
providing a first sample obtained from a subject before receiving allergen immunotherapy;
providing a second sample obtained from the subject who has received, or who is receiving, allergen immunotherapy;
wherein the first and second samples comprise B cells; and
determining the proportion or number of CD23+ allergen specific Bmem or CD23hi allergen specific Bmem in the first and second samples;
wherein an increase in the proportion or number of CD23+ allergen specific Bmem or CD23hi allergen specific Bmem in the second sample compared to the first sample indicates efficacy of an allergen immunotherapy in a subject.

In this aspect, the method further comprises determining the proportion or number of CD23+ allergen specific Bmem or CD23hi allergen specific Bmem expressing one or more additional biomarkers. The additional markers may be biomarkers that are described in Table 2 and/or Table 3. Preferably, the biomarkers are those that increase or decrease 1.5 fold, 2 fold, 3 fold or 4 fold as described in Table 2 and/or Table 3.

In another aspect, the present invention provides a method of determining efficacy of an allergen immunotherapy in a subject, the method comprising:
providing a first sample obtained from a subject before receiving allergen immunotherapy;
providing a second sample obtained from the subject who has received, or who is receiving, allergen immunotherapy;
wherein the first and second samples comprise B cells; and
determining the proportion or number of IL4Rα+ allergen specific Bmem or IL4Rαhi allergen specific Bmem in the first and second samples;
wherein an increase in the proportion or number of IL4Rα+ allergen specific Bmem or IL4Rαhi allergen specific Bmem in the second sample compared to the first sample indicates efficacy of an allergen immunotherapy in a subject.

In this aspect, the method further comprises determining the proportion or number of IL4Rα+ allergen specific Bmem or IL4Rαhi allergen specific Bmem expressing one or more additional biomarkers. The additional markers may be biomarkers that are described in Table 2 and/or Table 3. Preferably, the biomarkers are those that increase or decrease 1.5 fold, 2 fold, 3 fold or 4 fold as described in Table 2 and/or Table 3.

In any aspect or embodiment, the allergen specific B cells, preferably Bmem, are specific for the allergen included in the allergen immunotherapy that the subject had received or is receiving.

In any aspect or embodiment, the proportion of IgG+ Bmem or IgG4+ allergen specific Bmem is the relative number of IgG+ Bmem or IgG4+ allergen specific Bmem compared to the total number of Bmem or allergen specific Bmem, or the number of IgG+ Bmem or IgG4+ allergen specific Bmem cells, respectively, per microliter of blood. In any aspect or embodiment, IgG+ may include all IgG isotypes.

In any aspect or embodiment, the proportion of CD29+ allergen specific Bmem or CD29hi allergen specific Bmem is the relative number of CD29+ allergen specific Bmem or CD29hi allergen specific Bmem compared to the total number of allergen specific Bmem, or the number of CD29+ allergen specific Bmem or CD29hi allergen specific Bmem cells per microliter of blood.

In any aspect or embodiment, the proportion of CD23+ allergen specific Bmem or CD23hi allergen specific Bmem is the relative number of CD23+ allergen specific Bmem or CD23hi allergen specific Bmem compared to the total number of allergen specific Bmem, or the number of CD23+ allergen specific Bmem or CD23hi allergen specific Bmem cells per microliter of blood.

In any aspect or embodiment, the proportion of IL4Rα+ allergen specific Bmem or IL4Rαhi allergen specific Bmem is the relative number of IL4Rα+ allergen specific Bmem or IL4Rαhi allergen specific Bmem compared to the total number of allergen specific Bmem, or the number of IL4Rα+ allergen specific Bmem or IL4Rαhi allergen specific Bmem cells per microliter of blood.

The increase in the level or amount of one or more biomarkers in B-cells in a second sample compared to a first sample, may be accompanied by decreases in the level or amount of one or more biomarkers in the same, which indicates efficacy of an allergen immunotherapy in a subject.

In some embodiments of the present invention, the increase in the level or amount of one or more biomarkers may be of a fold-change of different magnitude, in comparison to the fold-change decrease in the level or amount of one or more biomarkers in B-cells in a second sample compared to a first sample, which indicates efficacy of an allergen immunotherapy in a subject.

In any aspect of the present invention, the second sample may be obtained from a subject at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 12 months, or any other time period defined herein, after initiation of the allergen immunotherapy. In some embodiments of the present invention, the second sample, or subsequent samples, may be obtained from a subject, between about 1 to 12 months, between about 2 to 12 months, between about 3 to 12 months, between about 4 to 12 months, between about 5 to 12 months, between about 6 to 12 months, between about 1 to 5 years, between about 2 to 5 years, between about 3 to 5 years, between about 4 to 5 years, or up to about 5 years, after initiation of the allergen immunotherapy.

In any aspect of the present invention, the second sample may be obtained from a subject at least 1 month, at least 2 months, at least 3 months, at least 4 months, at least 5 months, at least 6 months, at least 7 months, at least 8 months, at least 9 months, at least 10 months, at least 11 months, or at least 12 months after initiation of the allergen immunotherapy. In some embodiments of the present invention, the second sample, or subsequent samples, may be obtained from a subject, between 1 to 12 months, between 2 to 12 months, between 3 to 12 months, between 4 to 12 months, between 5 to 12 months, between 6 to 12 months, between 1 to 5 years, between 2 to 5 years, between 3 to 5 years, between 4 to 5 years, or up to 5 years, after initiation of the allergen immunotherapy.

In any aspect of the present invention, the second sample may be obtained from a subject 1 month, 2 months, 3 months, 4 months, 5 months, 6 months, 7 months, 8 months, 9 months, 10 months, 11 months, 12 months after initiation of the allergen immunotherapy.

In some embodiments of the present invention, the second sample may be obtained from a subject at least after the initiation phase of the allergen immunotherapy. For example, the initiation phase of allergen immunotherapy may be 4 months, or about 4 months, for SLIT for ryegrass pollen (RGP) or house dust mite (HDM) allergy, or 63 days, or about 63 days, for ultra-rush AIT for insect venom allergy.

In any aspect of the present invention, the allergen immunotherapy may be administered under the tongue (sublingually), orally, needle-free injections, by injections under the skin (subcutaneous) or in some instances administered by intradermal injection, or utilise the epicutaneous or intralymphatic administration routes. In a preferred embodiment of this aspect of the present invention, the allergen immunotherapy may be administered sublingually.

In any aspect of the present invention, the B cells may comprise or consist of B memory cells (Bmem). Preferably, the B cells are allergen-specific Bmem. In any embodiment, the Bmem may be identified as having the same, or similar, transcriptional profiles as clusters 3 and 4 as described in the Examples. Preferably, cluster 3 predominantly expresses IGHM, IGHG3 and IGHG1, and cluster 4 is enriched for IGHE, IGHG1 and/GHG4 expression. Cluster 3 may have increased expression of ITGB1 and PPP1R18 alongside decreased CXCR4 expression, and increased expression of FGR, SIGLEC10, CD99, TLE3 and decreased expression of TXNIP, BTG1 and FCER2, preferably compared to Bmem with the same, or similar, transcriptional profiles as cells identified as cluster 1 (see Examples). Cluster 4 may have increased expression of immune-related genes such as PARM1, IL13RA1, ITGB1, PPP1R18, SELL, FCER2 and decreased expression of CXCR4 and CD69, preferably compared to Bmem with the same, or similar, transcriptional profiles as cells identified as cluster 1 (see Examples). Cluster 4 Bmem may have an increased expression of SELL, FCER2, IGHE, IL13RA1, IL4R, PARM1 and ITGB1 whereas cluster 3 Bmem may have an increased expression of IGHM, IGHD, CD1C, PLAC8, SYK, FGR, FCGR2B, FCRLA, FCRL2, FCRL3, FCRL5 and ITGAX. Cluster 4 Bmem may have an expression patter of ITGB1, FCER2 and IL13RA1 not shared by cluster 1 to 3 Bmem.

In any aspect of the present invention, the sample may be a bodily fluid, for example, a whole blood sample, or serum. Alternatively, the same may be a tissue sample. The blood sample may be a whole blood, buffy coat, peripheral blood mononuclear cells (PBMC), cord blood, purified or sorted cell population or bodily fluid. Bodily fluids include lymph, semen, nasal secretions, bronchial secretions, alveolar fluid, cerebrospinal fluid, endolymph, synovial fluid, pleural fluid, pericardial fluid (pericardial liquor), menstrual fluid, or combinations thereof. The tissue sample may be selected from tonsil, lymph node, bronchial, nasal or gut or skin biopsy. The blood sample may be treated to prevent coagulation, e.g. with heparin or ethylenediaminetetraacetic acid (EDTA).

In a preferred embodiment, the sample comprises peripheral blood mononuclear cells, which may be enriched or purified for allergen-specific B cells. The process of enrichment or purification for allergen-specific B cells may occur via any suitable cell separation and isolation techniques, for example those that utilise cellular adhesion, density-based centrifugation, antibody-binding methods including fluorescence-activated cell sorting (FACS), magnetic-activated cell sorting (MACS) and aptamer binding, selective growth in culture, laser capture microdissection, or red blood cell rosetting. Most preferably enrichment or purification for allergen-specific B cells occurs via FACS.

In any aspect or embodiment, the biomarker is a protein, nucleic acid, for example RNA, or amplification product. Where the biomarker is a nucleic acid or amplification product, the method includes determining the level or amount of expression of the gene or RNA. Preferably, the biomarker is one or more nucleic acids comprising nucleotide sequences from genes or RNA transcripts of genes, where the genes are one or more of: IGHE, ITGB1, CD69, IL13RA1, CD99, IGHD, CXCR4, FCRL3, FCRL2, FCRL5, SIGLEC10, CD1C, FCER2, and IL4R.

In any aspect or embodiment described herein, where there is reference to a protein, the invention also includes determining or measuring the presence of, level of or amount of, as the case may be, the corresponding RNA (that was translated into the protein) or an amplification product thereof.

In some embodiments of the present invention, the method may further comprise determining the level or amount of one or more biomarkers in B-cells in the first and second samples, wherein the biomarkers are selected from the group consisting of IGHE, PPP1R18, ITGB1, PARM1, c-jun, CD69, IgE, phostensin and CD29, and wherein an increase in the level or amount of one or more biomarkers selected from IGHE, PPP1R18, ITGB1, PARM1, IgE, phostensin and CD29 in B-cells in the second sample compared to the first sample indicates efficacy of an allergen immunotherapy in a subject, and/or wherein a decrease in the level or amount of one or more biomarkers selected from c-jun and CD69 in B-cells in the second sample compared to the first sample indicates efficacy of an allergen immunotherapy in a subject. The biomarker may be nucleic acid, for example RNA or amplification product, or protein.

In any aspect of the present invention, the level or amount of one or more biomarkers may be the level or amount of RNA. Preferably, wherein the RNA is any one of pre-mRNA or mature mRNA, and wherein changes to level or amount of RNA may be determined using RNA sequencing. Most preferably, changes to level or amount of RNA may be determined using single-cell transcriptomics.

In any aspect of the present invention, allergen immunotherapy is for treatment of sensitisation to an allergen. The allergen may be a food-based allergen, airborne allergen, drug allergen, peptide allergen, goat milk allergen, plant allergen, animal allergen or arthropod allergen, wherein the arthropod may be an insect, myriapod, arachnid or crustacean (for example, insect, mite, crustacean).

Where the allergen may be food-based, it may include tree nuts, sesame, buckwheat, peanuts, milk proteins, egg whites, etc. Typical food allergens include milk allergens (Bos d 4, 5 and 8), peanut allergens (Ara h 1, 2, 3, 6, 8 and 9), hazelnut (Cor a 9 and 14), cashew nut (Ana o 3), Walnut (Jug r 1), Brazil nut (Ber e 1), Sesame (Ses i 1), Buckwheat (Fag e 3), almond (Pru du 6), peach (Pru p 1 and Pru p 3), shrimp (Pen m 1) and wheat (Tri a 19; omega-5-gliadin, Tri a 20, Tri a 21).

Where the allergen may be airborne, it may include grass pollens, tree pollen, weed pollen, animal danders, house dust mite feces, etc., and mold allergens.

Other common aeroallergens may include house dust mite feces from Dermatophagoides pteryonyssinus (Der p 1 and 2) or Dermatophagoides farina (Der f 1 and 2); pollen allergens from ryegrass (Lol p 1, 5), timothy grass (Phl p 1, 5), Bahia grass (Pas n 1), Bermuda grass (Cyn d 1), ragweed (Amb a 1), pellitory species (Par o 1; Par j 1, 2), birch (Bet v 1) and other atmospheric pollens including Olea europaea, Artemisia sp., gramineae, etc.; and animal dander, e.g. from cats (Fel d 1) and dogs (Can f 1).

Other allergens may include venom allergens from the honeybee (Api m 1, 3, 10); phospholipases from the yellow jacket Vespula maculifrons, common paper wasp Polistes exclamans and white-faced hornet Dolichovespula maculata and venom from jumper ant Myrmecia pilosula.

Other allergens may be derived from the Alternaria, Aspergillus and Cladosporium species for mold allergies, as well as allergic dermatitis caused by blood sucking arthropods, e.g. Diptera, including mosquitos (Anopheles sp., Aedes sp., Culiseta sp., Culex sp.); flies (Phlebotomus sp., Culicoides sp.) particularly black flies, deer flies and biting midges; ticks (Dermacenter sp., Ornithodoros sp., Otobius sp.); fleas, e.g. the order Siphonaptera, including the genera Xenopsylla, Pulex and Ctenocephalides.

In another aspect of the present invention, there is provided a kit for determining efficacy of an allergen immunotherapy in a subject comprising a means of detecting changes in the level or amount of transcripts, amplification products and/or proteins, wherein the transcripts, amplification products and/or proteins are selected from the group consisting any one or more of IGHE, ITGB1, CD69, IL13RA1, CD99, IGHD, CXCR4, FCRL3, FCRL2, FCRL5, SIGLEC10, CD1C, FCER2, IL4R or proteins: IgE, CD29, CD69, IL13Rα, CD99, IgD, CXCR4, FCRL3, FCRL2, FCRL5, SIGLEC10, CD1c, CD23, IL4Rα, and optionally PPP1R18, PARM1, phostensin, and c-jun, and wherein the kit may include written instructions.

In another aspect of the present invention, there is provided a method of measuring or detecting at least one biomarker, the method comprising: obtaining a sample from a subject after allergen immunotherapy, wherein the sample comprise B-cells; and measuring or detecting at least one biomarker or fragment thereof selected from the group consisting of IGHE, ITGB1, CD69, IL13RA1, CD99, IGHD, CXCR4, FCRL3, FCRL2, FCRL5, SIGLEC10, CD1C, FCER2, IL4R, PPP1R18, PARM1 and c-jun or proteins: IgE, CD29, CD69, IL13Rα, CD99, IgD, CXCR4, FCRL3, FCRL2, FCRL5, SIGLEC10, CD1c, CD23, IL4Rα and phostensin, or any combinations thereof in the sample.

In a preferred embodiment, measurement or detection of the at least one biomarker indicates that the subject has responded to the allergen immunotherapy that the subject has received, or is receiving. For example, where the at least one biomarker is selected from the group consisting of IGHE, ITGB1, IL13RA1, CD99, FCRL3, FCRL2, SIGLEC10, CD1C, FCER2, IL4R, PPP1R18, PARM1, IgE, CD29, IL13Rα, CD99, FCRL3, FCRL2, SIGLEC10, CD1c, CD23, IL4Rα and phostensin, or any combinations thereof, is not detectable, or detected at a lower level, in B-cells in a sample obtained from the subject prior to allergen immunotherapy, indicates that the subject has responded to the allergen immunotherapy.

Where the at least one biomarker is selected from the group consisting of CD69, IGHD, CXCR4, FCRL3, FCRL2, FCRL5, CD1C, FCER2, IL4R, c-jun, CD69, IgD, CXCR4, FCRL3, FCRL2, FCRL5, CD1c, CD23, IL4Rα, or any combinations thereof, is detectable, or detected at a higher level, in a sample obtained from the subject prior to allergen immunotherapy, indicates that the subject has responded to the allergen immunotherapy.

In one embodiment, increase in the level or amount of one or more of IGHE, ITGB1, IL13RA1, CD99, FCRL3, FCRL2, SIGLEC10, CD1C, FCER2, IL4R, PPP1R18, PARM1, IgE, CD29, IL13Rα, CD99, FCRL3, FCRL2, SIGLEC10, CD1c, CD23, IL4Rα and phostensin biomarkers in B-cells in a second sample compared to a first sample, may be accompanied by decreases in the level or amount of one or more of CD69, IGHD, CXCR4, FCRL3, FCRL2, FCRL5, CD1C, FCER2, IL4R, c-jun, CD69, IgD, CXCR4, FCRL3, FCRL2, FCRL5, CD1c, CD23 and IL4Rα in the same B cells in a second sample compared to a first sample, which indicates that the subject has responded to the allergen immunotherapy.

Preferably, levels of any one or more of IGHE, ITGB1, CD99, SIGLEC10, FCER2, IL4R, IgE, CD29, CD99, SIGLEC10, CD23, and IL4Rα, are higher in a second sample obtained from the subject during or after allergen immunotherapy, compared to levels of the same in a first sample obtained from the subject before allergen immunotherapy.

In one embodiment, levels of any one or more of CD69 and CD69 are lower in a second sample obtained from the subject during or after allergen immunotherapy, compared to levels of the same in a first sample obtained from the subject before allergen immunotherapy.

In one embodiment, increased levels of any one or more of IGHE, ITGB1, CD99, SIGLEC10, FCER2, IL4R, IgE, CD29, CD99, SIGLEC 10, CD23 and IL4Rα, may be accompanied by decreases in levels of any one or more of CD69 and CD69, when compared to levels of the same in a sample obtained from the subject before allergen immunotherapy.

In one embodiment, levels of any one or more of IGHE, ITGB1, IL13RA1, CD99, FCER2, IL4R, IgE, CD29, IL13Rα, CD99, CD23, and IL4Rα may be higher in a second sample obtained from the subject after allergen immunotherapy, compared to levels of the same in a first sample obtained from the subject before allergen immunotherapy. The increase in levels may be accompanied by decreases in any one or more of CD69, IGHD, CXCR4, FCRL3, FCRL2, FCRL5, CD1C, CD69, IgD, CXCR4, FCRL3, FCRL2, FCRL5 and CD1c in a second sample obtained from the subject after allergen immunotherapy, compared to levels of the same in a first sample obtained from the subject before allergen immunotherapy.

In one embodiment, levels of any one or more of ITGB1, CD99, FCRL3, FCRL2, SIGLEC10, CD1C, CD29, CD99, FCRL3, FCRL2, SIGLEC10, and CD1c, may be higher in a second sample obtained from a subject after allergen immunotherapy, compared to levels of the same in a first sample obtained from the subject before allergen immunotherapy. The increase in levels may be accompanied by decreases in any one or more of CD69, IGHD, CXCR4, FCRL5, FCER2, IL4R, CD69, IgD, CXCR4, FCRL5, CD23 and IL4Rα, in a second sample obtained from a subject after allergen immunotherapy, compared to levels of the same in a first sample obtained from the subject before allergen immunotherapy.

In another aspect of the present invention, there is provided a biomarker panel for determining efficacy of allergen immunotherapy of a subject, the panel comprising at least one of the following biomarkers: IGHE, ITGB1, CD69, IL13RA1, CD99, IGHD, CXCR4, FCRL3, FCRL2, FCRL5, SIGLEC10, CD1C, FCER2, IL4R, PPP1R18, PARM1 and c-jun or proteins: IgE, CD29, CD69, IL13Rα, CD99, IgD, CXCR4, FCRL3, FCRL2, FCRL5, SIGLEC10, CD1c, CD23, IL4Rα and phostensin, or any combinations thereof, wherein measurement or detection of the at least one biomarker indicates that the subject has responded to allergen immunotherapy.

In a preferred embodiment of this aspect of the present invention, there is provided a biomarker panel, wherein levels of any one or more of IGHE, ITGB1, IL13RA1, CD99, FCRL3, FCRL2, SIGLEC10, CD1C, FCER2, IL4R, PPP1R18, PARM1, IgE, CD29, IL13Rα, CD99, FCRL3, FCRL2, SIGLEC10, CD1c, CD23, IL4Rα and phostensin, are higher compared to levels of the same in a sample obtained from the subject before allergen immunotherapy; and/or wherein levels of any one or more of CD69, IGHD, CXCR4, FCRL3, FCRL2, FCRL5, CD1C, FCER2, IL4R, c-jun, CD69, IgD, CXCR4, FCRL3, FCRL2, FCRL5, CD1c, CD23 and IL4Rα, are lower compared to levels of the same in a sample obtained from the subject before allergen immunotherapy.

In another embodiment, there is provided a biomarker panel for determining efficacy of allergen immunotherapy of a subject, the panel comprising at least one of the following biomarkers: IGHE, ITGB1, CD69, CD99, SIGLEC10, FCER2, IL4R, IgE, CD29, CD69, CD99, SIGLEC10, CD23 and IL4Rα, or any combinations thereof, wherein measurement or detection of the at least one biomarker indicates that the subject has responded to allergen immunotherapy.

More preferably, the biomarker panel may detect increases in levels of any one or more of IGHE, ITGB1, CD99, SIGLEC10, FCER2, IL4R, IgE, CD29, CD99, SIGLEC 10, CD23 and IL4Rα, which may be accompanied by decreases in levels of any one or more of CD69 and CD69, when compared to levels of the same biomarkers in a sample obtained from the subject before allergen immunotherapy.

In another embodiment, there is provided a biomarker panel for determining efficacy of allergen immunotherapy of a subject, the panel comprising at least one of the following biomarkers: IGHE, ITGB1, CD69, IL13RA1, CD99, IGHD, CXCR4, FCRL3, FCRL2, FCRL5, CD1C, FCER2, IL4R, IgE, CD29, CD69, IL13Rα, CD99, IgD, CXCR4, FCRL3, FCRL2, FCRL5, CD1c, CD23, and IL4Rα, or any combinations thereof, wherein measurement or detection of the at least one biomarker indicates that the subject has responded to allergen immunotherapy.

More preferably, the biomarker panel may detect increases in levels of any one or more of IGHE, ITGB1, IL13RA1, CD99, FCER2, IL4R, IgE, CD29, IL13Rα, CD99, CD23, and IL4Rα, which may be accompanied by decreases in levels of any one or more of CD69, IGHD, CXCR4, FCRL3, FCRL2, FCRL5, CD1C, CD69, IgD, CXCR4, FCRL3, FCRL2, FCRL5 and CD1c, when compared to levels of the same biomarkers in a sample obtained from the subject before allergen immunotherapy.

In another embodiment, there is provided a biomarker panel for determining efficacy of allergen immunotherapy of a subject, the panel comprising at least one of the following biomarkers: ITGB1, CD69, CD99, IGHD, CXCR4, FCRL3, FCRL2, FCRL5, SIGLEC10, CD1C, FCER2, IL4R, CD29, CD69, CD99, IgD, CXCR4, FCRL3, FCRL2, FCRL5, SIGLEC10, CD1c, CD23, and IL4Rα, or any combinations thereof, wherein measurement or detection of the at least one biomarker indicates that the subject has responded to allergen immunotherapy.

More preferably, the biomarker panel may detect increases in levels of any one or more of ITGB1, CD99, FCRL3, FCRL2, SIGLEC10, CD1C, CD29, CD99, FCRL3, FCRL2, SIGLEC10, and CD1c, which may be accompanied by decreases in levels of any one or more of CD69, IGHD, CXCR4, FCRL5, FCER2, IL4R, CD69, IgD, CXCR4, FCRL5, CD23 and IL4Rα, when compared to levels of the same biomarkers in a sample obtained from the subject before allergen immunotherapy.

In certain embodiments, a panel of biomarkers is used for determining efficacy of allergen immunotherapy. Biomarker panels of any size can be used in the practice of the invention. In certain embodiments, the invention includes a biomarker panel comprising at least 3, at least 4, or at least 5, or at least 6, or at least 7, or at least 8, or at least 9, or at least 10, or at least 11, or at least 12, or at least 13, or at least 14, or more biomarkers. In one embodiment the biomarker panel comprises a means, such as a polynucleotide, probe, ligand, antibody, for detecting the presence of, increase of or decrease in the at least one or more biomarkers.

In another aspect, the invention provides a kit, panel or microarray comprising at least two diagnostic reagents described herein, each reagent identifying a different biomarker. In one embodiment, the kit comprises diagnostic reagents that bind to or complex individually with 2, 3, 4, 5, 6, 7, 8, 9, 10, 11, 12, 13, 14, 15, 16, 17, 18 or more biomarkers.

In any aspect of the present invention, the biomarker may be a unique peptide fragment or nucleic acid fragment thereof thereby serving as a surrogate of the full length protein or nucleic acid.

In any aspect of the present invention, the first and second samples are provided, or obtained, from the same subject. Alternatively, the first sample may be from a different subject or subjects who have the same or related allergy to the subject from which the second sample is provided or obtained and who have not received an allergen immunotherapy.

In any aspect, instead of providing a first sample and determining the level or amount of one or more biomarkers in B-cells in that first sample, a sample (i.e. test sample) obtained after the initiation of the allergen immunotherapy may be compared to reference values, or reference ranges, or a reference standard of the one or more biomarkers for subjects who have the same or related allergy and (a) who have not received an allergen immunotherapy, or (b) who have received an allergen immunotherapy. In this instance, where the level or amount of one or more biomarkers in B-cells in the test sample is the same or similar to reference values, or reference ranges, or a reference standard of the one or more biomarkers for subjects who have the same or related allergy and who have not received an allergen immunotherapy, then a determination can be made that the subject is not responding to the allergen immunotherapy. In this instance, where the level or amount of one or more biomarkers in B-cells in the test sample is different to reference values, or reference ranges, or a reference standard of the one or more biomarkers for subjects who have the same or related allergy and who have not received an allergen immunotherapy, then a determination can be made that the subject is responding to the allergen immunotherapy.

In this instance, where the level or amount of one or more biomarkers in B-cells in the test sample is different to reference values, or reference ranges, or a reference standard of the one or more biomarkers for subjects who have the same or related allergy and who have received an allergen immunotherapy, then a determination can be made that the subject is responding to the allergen immunotherapy. In this instance, where the level or amount of one or more biomarkers in B-cells in the test sample is different to reference values, or reference ranges, or a reference standard of the one or more biomarkers for subjects who have the same or related allergy and who have received an allergen immunotherapy, then a determination can be made that the subject is not responding to the allergen immunotherapy.

In any embodiment of the present invention the test sample may be obtained at least 1 month, at least 2 months, at least 3 months, at least 4 months, at least 5 months, at least 6 months, at least 7 months, at least 8 months, at least 9 months, at least 10 months, at least 11 months, or at least 12 months after initiation of the allergen immunotherapy. In some embodiments of the present invention, the test sample, or subsequent samples, may be obtained from a subject, between 6 to 12 months, or up to 5 years, after initiation of the allergen immunotherapy. In some embodiments of the present invention the test sample is obtained at least after the initiation phase of the allergen immunotherapy, for example 4 months for SLIT for RGP and HDM or 63 days for ultra-rush for insect venom allergy (in this instance there may not be a sample taken prior to treatment).

To perform a non-paired sample comparison, typically a normal reference range or reference standard will first be defined. Comparisons may then be conducted using the median expression level of particular biomarkers, as identified using fluorescence detection methods, or may involve analysis of the percentage of positive cells for one or more biomarkers.

In any aspect or embodiment of the present invention as described herein the step of determining the level or amount of one or more biomarkers in B-cells in a sample (e.g. a first or second sample) may instead be a step of determining the percentage of positive cells for one or more biomarkers in B-cells in a sample (e.g. a first or second sample). In addition, where an aspect or embodiment of the present invention contemplates an increase or decrease, as the case may be, of the level or amount of one or more biomarkers in B-cells in a sample compared to another sample (e.g. second sample compared to the first sample), that aspect or embodiment may also be performed by comparing an increase or decrease in the percentage of positive cells having one or more biomarkers. When comparing samples collected before and after allergen immunotherapy, the determining and comparison steps may involve an increase or decrease in the absolute or relative numbers of positive cells, which may be used as a measurement to indicate efficacy of an allergen immunotherapy in a subject. For example in this alternative aspect of the present invention, there is provided a method of determining efficacy of an allergen immunotherapy in a subject, the method comprising:
providing a first sample obtained from a subject before receiving allergen immunotherapy;
providing a second sample obtained from the subject who has received, or who is receiving, allergen immunotherapy;
wherein the first and second samples comprise B-cells; and
determining the percentage of positive cells having one or more biomarkers in B cells in the first and second samples, wherein the biomarkers are selected from the group consisting of IgE, CD29, CD69, IL13Rα, CD99, IgD, CXCR4, FCRL3, FCRL2, FCRL5, SIGLEC10, CD1c, CD23, and IL4Rα;
wherein an increase in the percentage of positive cells having one or more biomarkers, selected from IgE, CD29, IL13Rα, CD99, FCRL3, FCRL2, SIGLEC10, CD1c, CD23, and IL4Rα in B-cells in the second sample compared to the first sample indicates efficacy of an allergen immunotherapy in a subject, and/or
wherein a decrease in the percentage of positive cells having one or more biomarkers, selected from CD69, IgD, CXCR4, FCRL3, FCRL2, FCRL5, CD1c, CD23, IL4Rα in B-cells in the second sample compared to the first sample indicates efficacy of an allergen immunotherapy in a subject.

In any aspect or embodiment, the B-cells are Bmem cells (Bmem), more preferably IgG4+ Bmem. Typically, the Bmem are allergen specific Bmem, where the allergen is the same allergen provided in the allergen immunotherapy. Therefore, the Bmem may be allergen specific IgG4+ Bmem.

As used herein, except where the context requires otherwise, the term “comprise” and variations of the term, such as “comprising”, “comprises” and “comprised”, are not intended to exclude further additives, components, integers or steps.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1. Four months of SLIT increases Lol p 1-specific IgE and IgG4 in serum. A, Patient recruitment in May and June 2019 and timeline of 4 months of pre-seasonal SLIT for grass pollen allergy. B, Total and RGP-specific IgE in serum at timepoint 0. C, Lol p 1-specific IgE, IgG2 and IgG4 in serum at t=0 and 4 months. Statistical analysis was performed between baseline and timepoint 4 months using the Mann-Whitney U test; ** P<0.01 and **** P<0.0001, ns=not significant.

FIG. 2. Identification of B cells for single cell transcriptomics analysis. Single cell RNA sequencing of PBMC sorted for Lol p 1-specific Bmem by FACS from four patients before and after SLIT was conducted using the BD Rhapsody Whole Transcriptome Analysis pathway. A, Exemplary plot of Lol p 1-specific Bmem sorted for single cell transcriptomics. Number indicates proportion of Bmem. B, Genes with low expression or high and ubiquitous expression were excluded from analyses used for the identification of B cells due to the likelihood of insufficient sequencing and for removal of housekeeping genes. C, The top 200 genes with highest dispersion in expression amongst all cells were used to generate D, PCA plot with two distinct clusters. E, Heatmaps of expression patterns for canonical B cell gene MS4A1 and myeloid gene CD14 identified separate clusters. Monocytes (lower cluster) were excluded from further analyses.

FIG. 3. SLIT increases proportions of IGHE and IGHG4 transcripts in Lol p 1-specific memory B cells. A, Schematic of the constant genes in the human IGH locus depicting their position relative to the variable domain of the VDJ exon. B, Proportion of transcripts for IGH or IGK and IGL genes in single Lol p 1-specific Bmem before (n=189) and after SLIT (n=323) isolated from 4 allergic patients for single cell transcriptomic analysis. Cells arranged left-to-right based on maximal expression of IGH transcripts in the order of IGH genes in the human IGH locus (IGHM to IGHA2). C, Proportions of total IGH or IGHG gene transcripts in these Lol p 1-specific Bmem before and after SLIT. Significant differences in the proportional distribution of IGH and IGHG in (C) was determined by x² test; **** P<0.0001.

FIG. 4. SLIT increases the proportion of IGHE, IGHG1 and IGHG4-expressing Lol p 1-specific Bmem and promotes differentiation toward two transcriptionally distinct clusters. A, Differentially-expressed genes in Lol p 1-specific Bmem before and after SLIT. Genes with >1.5-fold change and q<0.05 identified by red arrows. B, Pseudotime clustering based on the 272 genes (Q<0.05) identified in 4A. C, B-cell clusters identified by pseudotime analysis before or after SLIT. D, Heat map of IGHM, IGHE and E, IGHG1-4 gene expression in B cells clustered by pseudotime analysis. F, IGH gene expression in Bmem from the four distinct pseudotime clusters. Significance determined by Mann-Whitney U test. * P<0.05, ** P<0.01, *** P<0.001 and **** P<0.0001, ns=not significant.

FIG. 5. Lol p 1-specific memory B cells generated by SLIT express ITGB1 (CD29), FCER2 (CD23) and IL13RA1 (CD213A1). A, Pseudotime clustering of all Lol p 1-specific Bmem from four patients before and after SLIT using genes identified in FIG. 3A. Numbers indicate proportion of clusters. B, Volcano plots of DEG comparing pseudotime clusters 1 to 4, 1 to 3 and 3 to 4. Significant genes with largest fold change or known immunological interest indicated by red arrows. C, Heatmap of gene expression for ITGB1, FCER2 and IL13RA1 on pseudotime plot from A.

FIG. 6. Increased numbers of circulating IgG4+ Lol p 1-specific Bmem and surface expression of CD29 and CD23 after SLIT. A, Number of circulating Lol p 1-specific Bmem and B, number of IgM+, IgG+, IgG4+ and IgA+ Lol p 1-specific Bmem from PBMC of RGP-allergic patients before and after SLIT (red lines; SLIT n=13) or from RGP-allergic patients that did not receive SLIT (blue lines; no SLIT n=14). The only patient to report no clinical benefit of SLIT is indicated by red dots and black lines. C, Representative histogram of CD29 expression on Lol p 1-specific Bmem from one RGP-allergic patient before (orange) and after (red) SLIT. Fluorescence minus one (FMO) control from after SLIT in grey. D, Number of CD29hi Lol p 1-specific Bmem and MFI of E, CD29, F, CD23 and G, CD213A1 on Lol p 1-specific Bmem. Significance determined by Mann-Whitney U test. * P<0.05 and ** P<0.01, ns=not significant.

FIG. 7. Increased expression of IL4Rα on allergen-specific Bmem after 4 months of SLIT. A, Example plot showing IL4Rα expression on Lol p 1+ Bmem from a patient with RGP allergy before (blue line; median 345) and 4 months after SLIT (red line; median 2262). Filled grey histogram is an unstained control (median 27.1). B, Frequencies of IL4Rα+ events within Lol p 1+ Bmem from 4 RGP allergic patients before and 4 months after SLIT. Statistics, paired t-test; **, p<0.01.

FIG. 8. Positive predictive values of biomarkers for prediction of outcome. A, Receiver-operator curves of ratios before/after 4 months for patients treated with SLIT vs untreated RGP allergic patients. Shown are Lol p 1+ Bmem numbers, CD29+ Lol p 1+ Bmem numbers and MFI of CD29 and CD213A1 on Lol p 1+ Bmem. B, Receiver-operator curves of data obtained at the 4-month timepoint for patients treated with SLIT vs untreated RGP allergic patients. Shown are Lol p 1+ Bmem numbers, CD29+ Lol p 1+ Bmem numbers and MFI of CD29 and CD213A1 on Lol p 1+ Bmem.

FIG. 9. Increased numbers of circulating IgG4+ Api m 1-specific Bmem and surface expression of CD29 and CD23 after ultra-rush AIT. A, Numbers of circulating Api m 1-specific Bmem and B, numbers of IgM+, IgG+, IgG4+ and IgA+ Api m 1-specific Bmem from PBMC of BV-allergic patients before and at day 63 after start of ultra-rush AIT (n=17). C, Representative histogram of CD29 expression on Api m 1-specific Bmem from one BV-allergic patient before (orange) and after (red) AIT. D, Numbers of CD29hi Api m 1-specific Bmem, and MFI of E, CD29, F, CD23 and G, CD213A1 on Api m 1-specific Bmem. Significance determined by Mann-Whitney U test. ** P<0.01, *** P<0.001, ns=not significant.

FIG. 10. No difference in numbers nor immunophenotype of total Bmem following AIT. A, number of IgM+, IgG+, IgG4+ and IgA+ Bmem from PBMC of RGP-allergic patients before and after SLIT (n=13). B, Number of CD29hi Lol p 1-specific Bmem and MFI of CD29, CD23 and CD213A1 on total Bmem. C, number of IgM+, IgG+, IgG4+ and IgA+ Bmem before and at day 63 after start of ultra-rush AIT (n=17). D, Number of CD29hi Lol p 1-specific Bmem and MFI of CD29, CD23 and CD213A1 on total Bmem. Significance determined by Mann-Whitney U test. * P<0.05 and ** P<0.01, ns=not significant.

### DETAILED DESCRIPTION OF THE EMBODIMENTS

It will be understood that the invention disclosed and defined in this specification extends to all alternative combinations of two or more of the individual features mentioned or evident from the text or drawings. All of these different combinations constitute various alternative aspects of the invention.

Further aspects of the present invention and further embodiments of the aspects described in the preceding paragraphs will become apparent from the following description, given by way of example and with reference to the accompanying drawings.

Reference will now be made in detail to certain embodiments of the invention. While the invention will be described in conjunction with the embodiments, it will be understood that the intention is not to limit the invention to those embodiments. On the contrary, the invention is intended to cover all alternatives, modifications, and equivalents, which may be included within the scope of the present invention as defined by the claims.

The present invention provides a method and a kit for determining treatment efficacy of allergen immunotherapy, and for measuring or detecting biomarkers in a subject undergoing allergen immunotherapy.

Advantages of aspects or embodiments of the invention is that the present method focuses on changes in levels or amounts of a selection of specific genes and/or proteins to provide rapid results that can efficiently assist with understanding efficacy of treatment when subjects undergo allergen immunotherapy. This can determine individuals that are responding from those that are not, allowing those that are not responding to be administered a different treatment or allergen immunotherapy. The invention therefore reduces the time during which an individual is receiving an allergen immunotherapy that may be of little or no benefit, thereby reducing the risk of adverse events associated with the allergen immunotherapy. In addition, the invention provides for increased treatment adherence as the subject can be made aware of treatment response. There may also be cost savings to patients in jurisdictions whereby allergen immunotherapy is not government subsidized. In such situations, many patients must make financial decisions as to which medications they can afford, therefore early identification of ineffective treatment would be advantageous.

### General

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or groups of compositions of matter. Thus, as used herein, the singular forms “a”, “an” and “the” include plural aspects, and vice versa, unless the context clearly dictates otherwise. For example, reference to “a” includes a single as well as two or more; reference to “an” includes a single as well as two or more; reference to “the” includes a single as well as two or more and so forth.

Those skilled in the art will appreciate that the present invention is susceptible to variations and modifications other than those specifically described. It is to be understood that the invention includes all such variations and modifications. The invention also includes all of the steps, features, compositions and compounds referred to or indicated in this specification, individually or collectively, and any and all combinations or any two or more of said steps or features.

One skilled in the art will recognize many methods and materials similar or equivalent to those described herein, which could be used in the practice of the present invention. The present invention is in no way limited to the methods and materials described.

All of the patents and publications referred to herein are incorporated by reference in their entirety.

The present invention is not to be limited in scope by the specific examples described herein, which are intended for the purpose of exemplification only. Functionally-equivalent products, compositions and methods are clearly within the scope of the present invention.

Any example or embodiment of the present invention herein shall be taken to apply mutatis mutandis to any other example or embodiment of the invention unless specifically stated otherwise.

Unless specifically defined otherwise, all technical and scientific terms used herein shall be taken to have the same meaning as commonly understood by one of ordinary skill in the art (for example, in cell culture, molecular genetics, immunology, immunohistochemistry, protein chemistry, and biochemistry).

Unless otherwise indicated, the recombinant protein, cell culture, and immunological techniques utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al. Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

The term “and/or”, e.g., “X and/or Y” shall be understood to mean either “X and Y” or “X or Y” and shall be taken to provide explicit support for both meanings or for either meaning.

As used herein the term “derived from” shall be taken to indicate that a specified integer may be obtained from a particular source albeit not necessarily directly from that source.

### Selected Definitions

As used herein, the term “allergen” refers to any naturally occurring protein or mixtures of proteins or chemicals/drugs that have been reported to induce allergic, i.e. IgE-mediated, reactions upon their repeated exposure to an individual.

An “allergy” also referred to herein as an “allergic reactivity,” is any condition where there is an undesired (e.g., a Type 1 hypersensitive) immune response (i.e., allergic response or reaction) to a substance. Such substances are referred to herein as allergens. Allergies or allergic conditions include, but are not limited to, allergic asthma, hay fever, hives, eczema, plant allergies, bee sting allergies, pet allergies, latex allergies, mold allergies, cosmetic allergies, food allergies, allergic rhinitis or coryza, topical allergic reactions, anaphylaxis, atopic dermatitis, hypersensitivity reactions and other allergic conditions. The allergic reaction may be the result of an immune reaction to any allergen. In some embodiments, the allergy is a food allergy. Food allergies include, but are not limited to, milk allergies, egg allergies, nut allergies, fish allergies, shellfish allergies, soy allergies or wheat allergies.

As used herein, the term “hypersensitivity” refers to an undesirable reaction produced by a normal immune response, including allergy and autoimmunity. This overreaction of the immune system may be damaging, uncomfortable or even fatal. Hypersensitivity reactions require a pre-sensitization of the host.

As used herein, the term “allergen sensitization” or “sensitization to an allergen” refers to the production of IgE antibodies following first exposure to an allergen or antigen that subsequently results in an allergic reaction or allergic reactivity.

As used herein, the term “immune cell” refers to any cell that is involved in an immune response. These cells include but are not limited to megakaryocyte, thrombocyte, erythrocyte, mast cell, myeloblast, basophil, neutrophil, eosinophil, monocyte, macrophage, dendritic cell, natural killer cell, NKT cells, NK-like cells, T cell, B cell and plasma cells.

The term “recombinant” shall be understood to mean the product of artificial genetic recombination. Accordingly, in the context of recombinant allergen or antigen, this term does not encompass a naturally-occurring allergen or antigen. However, if such an allergen or antigen is isolated, it is to be considered an isolated allergen or antigen. Similarly, if nucleic acid encoding the protein is isolated and expressed using recombinant means, the resulting protein is a recombinant allergen or antigen. A recombinant protein also encompasses a protein expressed by artificial recombinant means when it is within a cell, tissue or subject, e.g., in which it is expressed.

The term “ligand” or “reagent” refers, with regard to protein biomarkers, to a molecule that binds or complexes with a biomarker protein, molecular form or peptide, such as an antibody, antibody mimic or equivalent that binds to or complexes with a biomarker identified herein, a molecular form or fragment thereof. In certain embodiments, in which the biomarker expression is to be evaluated, the ligand can be a nucleotide sequence, e.g., polynucleotide or oligonucleotide, primer or probe.

As used herein, the term “antibody”, “immunoglobulin” or “lg” refers to a protein capable of specifically binding to one or a few closely related antigens by virtue of an antigen binding domain contained within a Fv. This term includes four chain antibodies (e.g., two light chains and two heavy chains), recombinant or modified antibodies (e.g., chimeric antibodies, humanized antibodies, human antibodies, CDR-grafted antibodies, primatized antibodies, de-immunized antibodies, synhumanized antibodies, half-antibodies, bispecific antibodies). An antibody generally comprises constant domains, which can be arranged into a constant region or constant fragment or fragment crystallizable (Fc). Exemplary forms of antibodies comprise a four-chain structure as their basic unit. Full-length antibodies comprise two heavy chains (˜50 to 70 kD) covalently linked and two light chains (˜23 kDa each). A light chain generally comprises a variable region (if present) and a constant domain and in mammals is either a k light chain or a λ light chain. A heavy chain generally comprises a variable region and one or two constant domain(s) linked by a hinge region to additional constant domain(s). Heavy chains of mammals are of one of the following types α, δ, ε, γ, or μ. Each light chain is also covalently linked to one of the heavy chains. For example, the two heavy chains and the heavy and light chains are held together by inter-chain disulfide bonds and by non-covalent interactions. The number of inter-chain disulfide bonds can vary among different types of antibodies. Each chain has an N-terminal variable region (VH or VL wherein each are ˜110 amino acids in length) and one or more constant domains at the C-terminus. The constant domain of the light chain (CL which is ˜110 amino acids in length) is aligned with and disulfide bonded to the first constant domain of the heavy chain (CH1 which is 330 to 440 amino acids in length). The light chain variable region is aligned with the variable region of the heavy chain. The antibody heavy chain can comprise 2 or more additional CH domains (such as, CH2, CH3 and the like) and can comprise a hinge region between the CH1 and CH2 constant domains. Antibodies can be of any type (e.g., IgG, IgE, IgM, IgD, IgA, and IgY), class (e.g., IgG1, IgG2, IgG3, IgG4, IgA1 and IgA2) or subclass. In one example, the antibody is a murine (mouse or rat) antibody or a primate (such as, human) antibody. In one example the antibody heavy chain is missing a C-terminal lysine residue. In one example, the antibody is humanized, synhumanized, chimeric, CDR-grafted or deimmunized.

The term “protein” shall be taken to include a single polypeptide chain, i.e., a series of contiguous amino acids linked by peptide bonds or a series of polypeptide chains covalently or non-covalently linked to one another (i.e., a polypeptide complex). For example, the series of polypeptide chains can be covalently linked using a suitable chemical or a disulphide bond. Examples of non-covalent bonds include hydrogen bonds, ionic bonds, Van der Waals forces, and hydrophobic interactions.

The term “polypeptide” or “polypeptide chain” will be understood from the foregoing paragraph to mean a series of contiguous amino acids linked by peptide bonds.

The term “microarray” refers to an ordered arrangement of binding/complexing array elements or ligands, e.g. antibodies, on a substrate.

The term “polynucleotide,” when used in singular or plural form, generally refers to any polyribonucleotide or polydeoxyribonucleotide, which may be unmodified RNA or DNA or modified RNA or DNA. Thus, for instance, polynucleotides as defined herein include, without limitation, single- and double-stranded DNA, DNA including single- and double-stranded regions, single- and double-stranded RNA, and RNA including single- and double-stranded regions, hybrid molecules comprising DNA and RNA that may be single-stranded or, more typically, double-stranded or include single- and double-stranded regions. In addition, the term “polynucleotide” as used herein refers to triple-stranded regions comprising RNA or DNA or both RNA and DNA. The term “polynucleotide” specifically includes cDNAs. The term includes DNAs (including cDNAs) and RNAs that contain one or more modified bases. In general, the term “polynucleotide” embraces all chemically, enzymatically and/or metabolically modified forms of unmodified polynucleotides, as well as the chemical forms of DNA and RNA characteristic of viruses and cells, including simple and complex cells.

The term “oligonucleotide” refers to a relatively short polynucleotide of less than 20 bases, including, without limitation, single-stranded deoxyribonucleotides, single- or double-stranded ribonucleotides, RNA: DNA hybrids and double-stranded DNAs. Oligonucleotides, such as single-stranded DNA probe oligonucleotides, are often synthesized by chemical methods, for example using automated oligonucleotide synthesizers that are commercially available. However, oligonucleotides can be made by a variety of other methods, including in vitro recombinant DNA-mediated techniques and by expression of DNAs in cells and organisms.

As used herein, the term “binds” in reference to the interaction of an allergen or antigen with an antibody means that the interaction is dependent upon the presence of a particular structure (e.g., an antigenic determinant or epitope) on the allergen or antigen. For example, an antibody recognizes and binds to a specific protein structure rather than to proteins generally. If an antibody binds to epitope “A”, the presence of a molecule containing epitope “A” (or free, unlabelled “A”), in a reaction containing labelled “A” and the protein, will reduce the amount of labelled “A” bound to the antibody.

As used herein, the term “epitope” (syn. “antigenic determinant”) shall be understood to mean a region of a protein (such as an allergen or antigen) to which an antigen binding domain of an antibody binds.

As used herein, the term “condition” refers to a disruption of or interference with normal function, and is not to be limited to any specific condition, and will include diseases or disorders.

“Diagnosis” as used herein generally includes determination as to whether a subject is likely affected by a given disease, disorder or dysfunction. The skilled artisan often makes a diagnosis on the basis of one or more diagnostic indicators, i.e., a biomarker, the presence, absence, or amount of which is indicative of the presence or absence of the disease, disorder or dysfunction.

“Prognosis” as used herein generally refers to a prediction of the probable course and outcome of a clinical condition or disease. A prognosis of a patient is usually made by evaluating factors or symptoms of a disease that are indicative of a favorable or unfavorable course or outcome of the disease. It is understood that the term “prognosis” does not necessarily refer to the ability to predict the course or outcome of a condition with 100% accuracy. Instead, the skilled artisan will understand that the term “prognosis” refers to an increased probability that a certain course or outcome will occur; that is, that a course or outcome is more likely to occur in a patient exhibiting a given condition, when compared to those individuals not exhibiting the condition.

As used herein, the term “subject” shall be taken to mean any animal including humans, for example a mammal. Exemplary subjects include but are not limited to humans and non-human primates. For example, the subject is a human.

“Reference standard” as used herein refers to the source of the reference biomarker levels. The “reference standard” is preferably provided by using the same assay technique as is used for measurement of the subject's biomarker levels in the reference subject or population, to avoid any error in standardization. The reference standard is, alternatively, a numerical value, a predetermined cutpoint, a mean, an average, a numerical mean or range of numerical means, a numerical pattern, a ratio, a graphical pattern, a protein abundance profile, protein level profile, median expression level or percentage of positive cells derived from the same biomarker or biomarkers in a reference subject or reference population. In an embodiment, in which expression of nucleic acid sequences encoding the biomarkers is desired to be evaluated, the reference standard can be an expression level of one or more biomarkers or an expression profile.

As used herein, the term “percentage of positive cells” shall be taken to mean a proportion of positive cells having one or more biomarkers, when comparing samples collected before and after allergen immunotherapy, which may be calculated by determining the increase or decrease in the absolute or relative numbers of positive cells. This measurement may be used in addition to, or as a substitute to, measurements for determining the increase or decrease in the level or amount of one more biomarkers, to indicate efficacy of an allergen immunotherapy in a subject.

### Methods of Determining Efficacy of an Allergen Immunotherapy

In one aspect, the present invention provides a method of determining efficacy of an allergen immunotherapy in a subject, the method comprising:
providing a first sample obtained from a subject before receiving allergen immunotherapy;
providing a second sample obtained from the subject who has received, or who is receiving, allergen immunotherapy;
wherein the first and second samples comprise B-cells; and
determining the level or amount of one or more biomarkers in B-cells in the first and second samples, or determining the percentage of positive cells having one or more biomarkers in B cells in the first and second samples, wherein the biomarkers are selected from the group consisting of IgE, CD29, CD69, IL13Rα, CD99, IgD, CXCR4, FCRL3, FCRL2, FCRL5, SIGLEC10, CD1c, CD23, and IL4Rα;
wherein an increase in the level or amount of one or more biomarkers, or an increase in the percentage of positive cells having one or more biomarkers, selected from IgE, CD29, IL13Rα, CD99, FCRL3, FCRL2, SIGLEC10, CD1c, CD23, and IL4Rα in B-cells in the second sample compared to the first sample indicates efficacy of an allergen immunotherapy in a subject, and/or
wherein a decrease in the level or amount of one or more biomarkers, or a decrease in the percentage of positive cells having one or more biomarkers, selected from CD69, IgD, CXCR4, FCRL3, FCRL2, FCRL5, CD1c, CD23, IL4Rα in B-cells in the second sample compared to the first sample indicates efficacy of an allergen immunotherapy in a subject.

In any aspect of the present invention, determining efficacy of an allergen immunotherapy may involve identifying increases or decreases in the level, amount of, or proportion of, one or more specific biomarkers. The increases or decreases may be fold-changes in expression levels of one or more specific biomarkers or may be represented by the percentage of positive cells (the frequencies of cells) having expression of one or more specific biomarkers, when comparing samples before and after allergen immunotherapy. The magnitude of the increase or decrease in the level, amount of, or proportion of, one or more specific biomarkers (as the context requires) may be any one described herein including in the Examples and Tables.

In one embodiment, the one or more biomarkers is one of more nucleic acids comprising nucleotide sequences from genes or RNA transcripts of genes, or amplification products thereof, where the genes are one or more of: IGHE, ITGB1, CD69, IL13RA1, CD99, IGHD, CXCR4, FCRL3, FCRL2, FCRL5, SIGLEC10, CD1C, FCER2 and IL4R.

In one embodiment of this aspect of the present invention, there may be at least a 1.5-fold increase in the level or amount of one or more biomarkers selected from IgE, CD29, IL13Rα, CD99, SIGLEC10, CD1c and CD23 in B-cells in a second sample compared to a first sample, which indicates efficacy of an allergen immunotherapy in a subject. The one or more biomarkers may be one of more nucleic acids comprising nucleotide sequences from genes or RNA transcripts of genes, or amplification products thereof, where the genes are one or more of: IGHE, ITGB1, IL13RA1, CD99, SIGLEC10, CD1C and FCER2.

In one embodiment of this aspect of the present invention, there may be at least a 2-fold increase in the level or amount of one or more biomarkers, this may include any one of more of IgE, CD29, IL13Rα, CD99, SIGLEC10, and CD1c in B-cells in a second sample compared to a first sample, which indicates efficacy of an allergen immunotherapy in a subject. The one or more biomarkers may be one of more nucleic acids comprising nucleotide sequences from genes or RNA transcripts of genes, or amplification products thereof, where the genes are one or more of: IGHE, ITGB1, IL13RA1, CD99, SIGLEC10 and CD1C.

In one embodiment of this aspect of the present invention, there may be at least a 2.5-fold increase in the level or amount of one or more biomarkers, this may include any one of more of IgE, CD29, IL13Rα, CD99 and IL4Rα in B-cells in a second sample compared to a first sample, which indicates efficacy of an allergen immunotherapy in a subject. The one or more biomarkers may be one of more nucleic acids comprising nucleotide sequences from genes or RNA transcripts of genes, or amplification products thereof, where the genes are one or more of: IGHE, ITGB1, IL13RA1 and CD99.

In one embodiment of this aspect of the present invention, there may be at least a 3-fold increase in the level or amount of one or more biomarkers, this may include any one of more of IgE, CD29, and IL13Rα in B-cells in a second sample compared to a first sample, which indicates efficacy of an allergen immunotherapy in a subject. The one or more biomarkers may be one of more nucleic acids comprising nucleotide sequences from genes or RNA transcripts of genes, or amplification products thereof, where the genes are one or more of: IGHE, ITGB1 and IL13RA1.

In one embodiment of this aspect of the present invention, there may be at least a 4-fold increase in the level or amount of a biomarker, this may include CD29 in B-cells in a second sample compared to a first sample, which indicates efficacy of an allergen immunotherapy in a subject. The biomarker may be a nucleic acid comprising nucleotide sequences from the gene or RNA transcripts of the gene, or amplification product thereof, where the gene is ITGB1.

In one embodiment of this aspect of the present invention, there may be at least a 1.5-fold decrease in the level or amount of one or more of CD69, IgD, CXCR4, FCRL3, FCRL2, FCRL5, CD1c, CD23 and IL4Rα in B-cells in a second sample compared to a first sample, which indicates efficacy of an allergen immunotherapy in a subject. The one or more biomarkers may be one of more nucleic acids comprising nucleotide sequences from genes or RNA transcripts of genes, or amplification products thereof, where the genes are one or more of: CD69, IGHD, CXCR4, FCRL3, FCRL2, FCRL5, CD1C, FCER2 and IL4R.

In one embodiment of this aspect of the present invention, there may be at least a 2-fold decrease in the level or amount of one or more biomarkers, this may include any one of more of CD69, IgD, CXCR4, FCRL3, FCRL2, FCRL5, CD23 and IL4Rα in B-cells in a second sample compared to a first sample, which indicates efficacy of an allergen immunotherapy in a subject. The one or more biomarkers may be one of more nucleic acids comprising nucleotide sequences from genes or RNA transcripts of genes, or amplification products thereof, where the genes are one or more of: CD69, IGHD, CXCR4, FCRL3, FCRL2, FCRL5, FCER2 and IL4R.

In one embodiment of this aspect of the present invention, there may be at least a 2.5-fold decrease in the level or amount of one or more biomarkers, this may include any one of more of CD69, IgD, CXCR4 and FCRL3 in B-cells in a second sample compared to a first sample, which indicates efficacy of an allergen immunotherapy in a subject. The one or more biomarkers may be one of more nucleic acids comprising nucleotide sequences from genes or RNA transcripts of genes, or amplification products thereof, where the genes are one or more of: CD69, IGHD, CXCR4 and FCRL3.

In one embodiment of this aspect of the present invention, there may be at least a 3-fold decrease in the level or amount of one or more biomarkers, this may include any one of more of IgD and CXCR4 in B-cells in a second sample compared to a first sample, which indicates efficacy of an allergen immunotherapy in a subject. The one or more biomarkers may be one of more nucleic acids comprising nucleotide sequences from genes or RNA transcripts of genes, or amplification products thereof, where the genes are one or more of: IGHD and CXCR4,

In one embodiment of this aspect of the present invention, there may be at least a 4-fold decrease in the level or amount of one or more biomarkers, this may include any one of more of IgD and CXCR4 in B-cells in a second sample compared to a first sample, which indicates efficacy of an allergen immunotherapy in a subject. The one or more biomarkers may be one of more nucleic acids comprising nucleotide sequences from genes or RNA transcripts of genes, or amplification products thereof, where the genes are one or more of: IGHD and CXCR4.

In another aspect, the present invention provides a method of measuring or detecting at least one biomarker, the method comprising:
obtaining a sample from a subject after allergen immunotherapy, wherein the sample comprise B-cells; and
measuring or detecting at least one biomarker or fragment thereof selected from the group consisting of IGHE, ITGB1, CD69, IL13RA1, CD99, IGHD, CXCR4, FCRL3, FCRL2, FCRL5, SIGLEC10, CD1C, FCER2, IL4R, PPP1R18, PARM1, c-jun or IgE, CD29, CD69, IL13Rα, CD99, IgD, CXCR4, FCRL3, FCRL2, FCRL5, SIGLEC10, CD1c, CD23, IL4Rα and phostensin, or any combinations thereof in the sample.

In one embodiment, the measurement or detection of the at least one biomarker indicates that the subject has responded to the allergen immunotherapy that the subject has received, or is receiving.

In one embodiment, the at least one biomarker or fragment thereof is selected from the group consisting of IGHE, ITGB1, IL13RA1, CD99, FCRL3, FCRL2, SIGLEC10, CD1C, FCER2, IL4R, PPP1R18, PARM1, IgE, CD29, IL13Rα, CD99, FCRL3, FCRL2, SIGLEC10, CD1c, CD23, IL4Rα and phostensin, or any combinations thereof; and wherein the at least one biomarker is not detectable, or detected at a lower level, in B-cells in a sample obtained from the subject prior to allergen immunotherapy.

In one embodiment, the at least one biomarker or fragment thereof is selected from the group consisting of CD69, IGHD, CXCR4, FCRL3, FCRL2, FCRL5, CD1C, FCER2, IL4R, c-jun, CD69, IgD, CXCR4, FCRL3, FCRL2, FCRL5, CD1c, CD23, IL4Rα, or any combinations thereof; and wherein the at least one biomarker is detectable, or detected at a higher level, in a sample obtained from the subject prior to allergen immunotherapy.

In one embodiment, the least one biomarker or fragment thereof is selected from the group consisting of IGHE, ITGB1, CD69, IL13RA1, CD99, IGHD, CXCR4, FCRL3, FCRL2, FCRL5, SIGLEC10, CD1C, FCER2, IL4R, PPP1R18, PARM1, c-jun, IgE, CD29, CD69, IL13Rα, CD99, IgD, CXCR4, FCRL3, FCRL2, FCRL5, SIGLEC10, CD1c, CD23, IL4Rα and phostensin, or any combinations thereof;

In one embodiment, the levels of any one or more of IGHE, ITGB1, IL13RA1, CD99, FCRL3, FCRL2, SIGLEC10, CD1C, FCER2, IL4R, PPP1R18, PARM1, IgE, CD29, IL13Rα, CD99, FCRL3, FCRL2, SIGLEC10, CD1c, CD23, IL4Rα and phostensin, are higher compared to levels of the same in a sample obtained from the subject before allergen immunotherapy; and/or the levels of one or more of CD69, IGHD, CXCR4, FCRL3, FCRL2, FCRL5, CD1C, FCER2, IL4R, c-jun, CD69, IgD, CXCR4, FCRL3, FCRL2, FCRL5, CD1c, CD23 and IL4Rα, are lower compared to levels of the same in a sample obtained from the subject before allergen immunotherapy.

In one embodiment, the levels of any one or more of IGHE, ITGB1, CD99, SIGLEC10, FCER2, IL4R, IgE, CD29, CD99, SIGLEC10, CD23, and IL4Rα, are higher in a second sample obtained from the subject after allergen immunotherapy, compared to levels of the same in a first sample obtained from the subject before allergen immunotherapy.

In one embodiment, the levels of any one or more of CD69 and CD69 are lower in a second sample obtained from the subject after allergen immunotherapy, compared to levels of the same in a first sample obtained from the subject before allergen immunotherapy.

In one embodiment, the at least one biomarker or fragment thereof is selected from the group consisting of IGHE, ITGB1, CD69, CD99, SIGLEC10, FCER2, IL4R, IgE, CD29, CD69, CD99, SIGLEC10, CD23 and IL4Rα, or combinations thereof.

In one embodiment, the levels of any one or more of IGHE, ITGB1, CD99, SIGLEC10, FCER2, IL4R, IgE, CD29, CD99, SIGLEC10, CD23 and IL4Rα are higher compared to levels of the same in a sample obtained from the subject before allergen immunotherapy; and/or the levels of any one or more of CD69 and CD69 are lower compared to levels of the same in a sample obtained from the subject before allergen immunotherapy.

In one embodiment, the levels of any one or more of IGHE, ITGB1, IL13RA1, CD99, FCER2, IL4R, IgE, CD29, IL13Rα, CD99, CD23, and IL4Rα are higher in a second sample obtained from the subject after allergen immunotherapy, compared to levels of the same in a first sample obtained from the subject before allergen immunotherapy.

In one embodiment, the levels of any one or more of CD69, IGHD, CXCR4, FCRL3, FCRL2, FCRL5, CD1C, CD69, IgD, CXCR4, FCRL3, FCRL2, FCRL5 and CD1c are lower in a second sample obtained from the subject after allergen immunotherapy, compared to levels of the same in a first sample obtained from the subject before allergen immunotherapy.

In one embodiment, the at least one biomarker or fragment thereof is selected from the group consisting of IGHE, ITGB1, CD69, IL13RA1, CD99, IGHD, CXCR4, FCRL3, FCRL2, FCRL5, CD1C, FCER2, IL4R, IgE, CD29, CD69, IL13Rα, CD99, IgD, CXCR4, FCRL3, FCRL2, FCRL5, CD1c, CD23, and IL4Rα or combinations thereof.

In one embodiment, the levels of any one of more of IGHE, ITGB1, IL13RA1, CD99, FCER2, IL4R, IgE, CD29, IL13Rα, CD99, CD23, and IL4Rα are higher compared to levels of the same in a sample obtained from the subject before allergen immunotherapy; and/or the levels of any one of more of CD69, IGHD, CXCR4, FCRL3, FCRL2, FCRL5, CD1C, CD69, IgD, CXCR4, FCRL3, FCRL2, FCRL5 and CD1c are lower compared to levels of the same in a sample obtained from the subject before allergen immunotherapy.

In one embodiment, the levels of any one or more of ITGB1, CD99, FCRL3, FCRL2, SIGLEC10, CD1C, CD29, CD99, FCRL3, FCRL2, SIGLEC10, and CD1c, are higher in a second sample obtained from a subject after allergen immunotherapy, compared to levels of the same in a first sample obtained from the subject before allergen immunotherapy.

In one embodiment, the levels of any one or more of CD69, IGHD, CXCR4, FCRL5, FCER2, IL4R, CD69, IgD, CXCR4, FCRL5, CD23 and IL4Rα, are lower in a second sample obtained from a subject after allergen immunotherapy, compared to levels of the same in a first sample obtained from the subject before allergen immunotherapy.

In one embodiment, the at least one biomarker or fragment thereof is selected from the group consisting of ITGB1, CD69, CD99, IGHD, CXCR4, FCRL3, FCRL2, FCRL5, SIGLEC10, CD1C, FCER2, IL4R, CD29, CD69, CD99, IgD, CXCR4, FCRL3, FCRL2, FCRL5, SIGLEC10, CD1c, CD23, and IL4Rα, or combinations thereof.

In one embodiment, the levels of any one or more of ITGB1, CD99, FCRL3, FCRL2, SIGLEC10, CD1C, CD29, CD99, FCRL3, FCRL2, SIGLEC10, and CD1c are higher compared to levels of the same in a sample obtained from the subject before allergen immunotherapy; and/or the levels of any one or more of CD69, IGHD, CXCR4, FCRL5, FCER2, IL4R, CD69, IgD, CXCR4, FCRL5, CD23 and IL4Rα are lower compared to levels of the same in a sample obtained from the subject before allergen immunotherapy.

In any aspect, the biomarkers are those that are described in Table 2 and/or Table 3. In any aspect, the biomarkers are those that increase or decrease 1.5 fold, 2 fold, 3 fold or 4 fold as described in Table 2 and/or Table 3.

### Allergy and Sensitization

It is understood that a method of the present invention has several important applications, in particular in the area of medical diagnostics of humans.

Hypersensitivity reactions due to immunological responses can be classified into four broad classes. In particular, type I hypersensitivity reaction are those immediate type allergic reactions mediated by IgE-antibodies. In most allergies, such as those to food, pollen and house dust mites, reactions occur because the subject has become sensitised to an innocuous antigen—the allergen—by producing IgE antibodies against it. Subsequent exposure of the allergen triggers the activation of IgE binding cells, including mast cells and basophils, in the tissue or blood, leading to a series of responses that are characteristic of this type of reaction including degranulation of effector cells, release of histamines, heparin, eosinophil and neutrophil chemotactic factors, leukotrienes and thromboxane etc. Allergic immune responses are those characterised by the production of high levels of IgE antibody, which may be detected in the blood, and production of IgE-specific B cells.

The conventional tests for hypersensitivity include a skin prick test, where the allergen is injected intracutaneously or, occasionally, intradermally. A hypersensitivity or allergic response will cause rapid production of a wheal and erythema within 30 minutes. Other tests of allergy are known to the skilled person in the art and include immunoassays tests such as enzyme-linked immunosorbent assay (ELISA, or EIA) and radioallergosorbent test (RAST). The ELISA test measures the amount of allergen-specific antibodies in the blood and RAST test looks for specific allergen-related antibodies in order to identify your allergy triggers.

A test of the present invention may be used for monitoring allergic reactivity or allergen sensitivity in circumstances where a skin prick test may not be warranted for example, (i) when the patient is using medicine known to interfere with the skin prick test such as antihistamines, corticosteroids or certain antidepressants, (ii) the subject cannot tolerate many needle scratches that are required for the skin testing, (iii) the subject has an unstable heart condition, (iv) poorly controlled asthma, severe eczema, dermatitis, psoriasis or other severe skin condition and/or (v) might have an extreme reaction during skin testing or have a history of life-threatening allergic reactions e.g. anaphylaxis.

Types of allergies include but are not limited to food allergy, skin allergy, dust mite or pollen allergy, insect sting allergy, pet allergy, eye allergy, drug allergy, allergic rhinitis, latex allergy in particular Type I IgE-mediated allergic reaction, mold allergy, allergy associated sinus infection and cockroach allergy. Food allergies include but are not limited to allergies to milk, egg, peanut, tree nut, soy, wheat, fish and shellfish. Drug allergies includes those which are IgE mediated by reacting to substances. The most common drug allergies include penicillin and other related antibiotics, antibiotics containing sulphonamides, anticonvulsants, aspirin, ibuprofen and other nonsteroidal anti-inflammatory drugs (NSAIDs) and chemotherapy drugs. The most common skin allergies include eczema (atopic dermatitis), hives (urticaria) and contact dermatitis. The most common forms of eye allergy are triggered by outdoor allergens, such as pollens from grass, trees and weeds, indoor allergens such as pet dander, dust mites and mold, irritants such as cigarette smoke, perfume and diesel exhaust. The most common dust or pollen allergy include dust mites, cockroaches, mold, pollen (e.g. grass, tree or weed), pet hair, fur or feathers. The type of symptoms of an allergic reaction include but are not limited to excess mucus production, loss of sense of smell or taste, sore throat and/or cough, tiredness, temperature or shivers, facial congestion, headache, toothache, post nasal drip, wheezing, shortness of breath, trouble breathing, throat and mouth swelling, nausea, vomiting, bloating, diarrhea, stomach pain, cramping abdominal pain, skin rash, itching (in particular of the nose, eyes, ears and mouth), red and watery eyes, swelling around the eyes, hives, swelling of the lips, tongue or throat, high blood pressure, dizziness and/or fainting, severe asthma episode (asthma attack), chronic asthma and anaphylaxis.

The subject may be hypersensitive or exhibit allergy to one or more allergens as described herein. Allergens include food-based allergens such as tree nuts, sesame, buckwheat, peanuts, milk proteins, egg whites, shrimp etc. Other allergens of interest include various airborne antigens, such as grass pollens, animal danders, house dust mite feces, etc., as well as insect venoms, and mold allergens. Typical food allergens include milk allergens (Bos d 4, 5 and 8), peanut allergens (Ara h 1, 2, 3, 6 and 8), hazelnut (Cor a 9 and 14), cashew nut (Ana o 3), Walnut (Jug r 1), Brazil nut (Ber e 1), Sesame (Ses i 1), Buckwheat (Fag e 3), almond (Pru du 6), black tiger shrimp (Pen m 1) and wheat (Tri a 19). Common aeroallergens include Dermatophagoides pteryonyssinus (Der p 1 and 2) or Dermatophagoides farina (Der f 1 and 2); pollen allergens from ryegrass (Lol p 1, 5), timothy grass (Phl p 1, 5), Bahia grass (Pas n 1), Bermuda grass (Cyn d 1), ragweed (Amb a 1), pellitory species (Par o 1; Par j 1, 2), birch (Bet v 1) and other atmospheric pollens including Olea europaea, Artemisia sp., gramineae, etc.; and animal dander, e.g. from cats (Fel d 1) and dogs (Can f 1). Other allergens include venom allergens from the honey bee (Api m 1, 3, 10); phospholipases from the yellow jacket Vespula maculifrons, common paper wasp Polistes exclamans and white-faced hornet Dolichovespula maculata and venom from jumper ant Myrmecia pilosula. Other allergens of interest are those responsible for mould allergies (esp from the Alternaria, Aspergillus and Cladosporium species), as well as allergic dermatitis caused by blood sucking arthropods, e.g. Diptera, including mosquitos (Anopheles sp., Aedes sp., Culiseta sp., Culex sp.); flies (Phlebotomus sp., Culicoides sp.) particularly black flies, deer flies and biting midges; ticks (Dermacenter sp., Ornithodoros sp., Otobius sp.); fleas, e.g. the order Siphonaptera, including the genera Xenopsylla, Pulex and Ctenocephalides. The allergen may be from a bacterium, for example, protein MGL_1304, which is secreted by the bacterium Malassezia (M.) globose, and is a major allergen for sweat allergy.

In relation to subjects with ryegrass pollen (RGP) allergy, they may have moderate to severe seasonal allergic rhinitis (SAR). The subject may or may not have asthma. In one embodiment, the subject may have serum RGP-specific IgE of ≥0.35 KUA/L.

In relation to subjects with bee venom allergy (BV allergy), they may be diagnosed on the basis of a systemic allergic response to a bee sting. In one embodiment, the subject may have serum BV-specific IgE of ≥0.35 kUA/L.

### Reagents and Ligands for Detecting Biomarkers

Any aspect of the present invention involves determining the level or amount of one or more biomarkers in B-cells. This may be performed by measuring or detecting the presence of, level of or expression of one or more biomarkers described herein. That may be performed by a reagent or ligand that binds to or complexes with a biomarker of Table 2 or a unique peptide thereof, or a molecular form thereof or a combination of such ligands.

In one embodiment, such a ligand or reagent desirably binds to a protein biomarker or a unique peptide contained therein, and can be an antibody which specifically binds a single biomarker of Table 2, or a unique peptide in that single biomarker. Various forms of antibody, e.g., polyclonal, monoclonal, recombinant, chimeric, as well as fragments and components (e.g., CDRs, single chain variable regions, etc.) or antibody mimics or equivalents may be used in place of antibodies. The ligand or reagent itself may be labelled or immobilized.

Examples of antibodies that binds to the biomarkers in Table 2, are included in Table 1 below:

TABLE 1
List of biomarkers and exemplary antibodies
for binding the biomarkers listed in Table 3.

| | | UniProt | |
|---|---|---|---|
| Number | Protein | identifier | Exemplary antibodies |
| 1 | IgE | P01854 | Anti-IgE (polyclonal; ThermoFisher); |
| | | | anti-IgE (BioLegend) |
| 2 | ITGB1 | P05556 | CD29 (TS2/16; BioLegend) |
| 3 | CD69 | Q07108 | CD69 (FN50; BioLegend) |
| 4 | IL13Rα | P78552 | CD213a1 (SS12B; BioLegend); |
| | | | CD213a1 (419718; R&D Systems) |
| 5 | CD99 | P14209 | CD99 (hec2; BioLegend) |
| 6 | IgD | P01880 | IgD (IA6-2; BD Biosciences); IgD |
| | | | (IA6-2; BioLegend) |
| 7 | CXCR4 | P61073 | CD184 (12G5; BD Biosciences); |
| | | | CD184 (QA18A64; BioLegend) |
| 8 | FCRL3 | Q96P31 | CD307c (H5; BioLegend) |
| 9 | FCRL2 | Q96LA5 | CD307b (REA474; Miltenyi Biotec) |
| 10 | FCRL5 | Q96RD9 | CD307e (509f6; BioLegend) |
| 11 | SIGLEC10 | Q96LC7 | Anti-human SIGLEC10 (5G6; |
| | | | BioLegend) |
| 12 | CD1c | P29017 | CD1c (L161; BioLegend) |
| 13 | FcεR2 | P06734 | CD23-APC (EBVCS-5; BioLegend) |
| 14 | IL4Rα | P24394 | CD124 (hIL4-M57; BD Biosciences) |
| | | | CD124 (G077F6; BioLegend) |

Any combination of labelled or immobilized biomarker reagents or ligands can be assembled in a diagnostic kit or device for the purposes of determining the efficacy of immunotherapy.

Thus, a kit or device can contain multiple reagents or one or more individual reagents. For example, one embodiment of a composition includes a substrate upon which the biomarkers or ligands are immobilized. In another embodiment, the kit also contains optional detectable labels, immobilization substrates, optional substrates for enzymatic labels, as well as other laboratory items.

The diagnostic reagents, devices, or kits compositions based on the biomarkers of Table 2 or fragments thereof described herein, optionally associated with detectable labels, can be presented in the format of a microfluidics card, a chip or chamber, a bead or a kit adapted for use with assays formats such as sandwich ELISAs, multiple protein assays, platform multiplex ELISAs, such as the BioRad Luminex platform, Mass spectrometry quantitative assays, or flow cytometry.

In another embodiment, the reagent ligands are nucleotide sequences, the diagnostic reagent is a polynucleotide or oligonucleotide sequence that hybridizes to gene, gene fragment, gene transcript or nucleotide sequence encoding a biomarker of Table 2 or encoding a unique peptide thereof. Such a polynucleotide/oligonucleotide can be a probe or primer, and may itself be labeled or immobilized. In one embodiment, ligand-hybridizing polynucleotide or oligonucleotide reagent(s) are part of a primer-probe set, and the kit comprises both primer and probe. Each said primer-probe set amplifies a different gene, gene fragment or gene expression product that encodes a different biomarker of Table 2. For use in the compositions the PCR primers and probes are preferably designed based upon intron sequences present in the biomarker gene(s) to be amplified selected from the gene expression profile. The design of the primer and probe sequences is within the skill of the art once the particular gene target is selected. The particular methods selected for the primer and probe design and the particular primer and probe sequences are not limiting features of these compositions.

In general, optimal PCR primers and probes used in the compositions described herein are generally 17-30 bases in length, and contain about 20-80%, such as, for example, about 50-60% G+C bases. Melting temperatures of between 5° and 80° C., e.g. about 50 to 70° C. are typically preferred.

The present methods of the invention involve detection of biomarkers as described herein.

The term “detectable” as used herein refers to an occurrence of, or a change in, a signal that is directly or indirectly detectable either by observation or by instrumentation. Typically, the detectable response is an occurrence of a signal wherein the fluorophore is inherently fluorescent. Alternatively, the detectable response is an optical response resulting in a change in the wavelength distribution patterns or intensity of absorbance or fluorescence or a change in light scatter, fluorescence lifetime, fluorescence polarization, or a combination of the above parameters. Other detectable responses include, for example, chemiluminescence, phosphorescence, radiation from radioisotopes, magnetic attraction, and electron density.

The term “label,” as used herein, refers to a chemical moiety or protein that is directly or indirectly detectable (e.g. due to its spectral properties, conformation or activity) when attached to a reagent or ligand and used in the present methods.

A detection label conjugated to a reagent or ligand may be any label that allows separate detection and quantitation, by flow cytometry. For example, a fluorochrome. Suitable fluorescent labels are known in the art and include fluorescein isothiocyanate (FITC), phycoerythrin (PE), peridin chlorophyll protein (PerCP), allophycocyanin (APC), Alexa fluor 488, Alexa fluor 647, Alexa fluor 710, Alexa fluor 405, cyanin 5 (Cy5), Cyanin 5.5 (Cy5.5), pacific blue (PacB), horizon violet 450 (HV450), pacific orange (PacO), horizon-V500 (HV500), Krome Orange, Brilliant Violet 421 (BV421), Brilliant Violet 510 (BV510), Brilliant Violet 605 (BV605), Brilliant Violet 650 (BV650), Brilliant Violet 711 (BV711), Brilliant Violet 785 (BV785), Brilliant Ultraviolet 395 (BUV395), Brilliant Ultraviolet 496 (BUV496), Brilliant Ultraviolet 737(BUV737), Orange Cytognos (OC) 515, quantum dots and conjugates thereof coupled with PE, to APC or to PerCP (e.g. PE/Cy5, PE/Cy5.5, PE/Cy7, PerCP/Cy5.5, APC/Cy7, APC-H7, APC-Alex750, PE-Texas Red, PE-Dazzle, PE-CF594), Cytek cFluor (e.g cFluor V420, V450, V547, B515, B532, BYG575, BYF610, BYG666, BYG690, BYG710, BYG781, R659, R720, R780) or any additional compatible fluorochrome or fluorochrome tandem, etc.

In one example, the antibodies are conjugated to (1) pacific blue (PacB), brilliant violet 421 (BV421) or Horizon V450; (2) pacific orange (PacO), Horizon V500 (HV500), BV510, Khrome orange (KO) or OC515, (3) Horizon BB515, fluorescein isothiocyanate (FITC) or Alexa488, (4) phycoerythrin (PE), (5) peridinin chlorophyl protein/cyanine 5.5 (PerCP-Cy5.5), PerCP or PE-TexasRed, (6) phycoerythrin/cyanine7 (PE-Cy7), (7) allophycocyanine (APC) or Alexa647, and (8) allophycocyanine/hilite 7 (APC-H7), APC-Cy7, Alexa680, APC-A750, APC-C750 or Alexa700.

In another example, the antibodies are conjugated to (1) brilliant violet 421, (2) brilliant violet 510 (BV510), (3) brilliant violet 650 (BV650), (4) brilliant violet 786 (BV786), (5) fluorescein isothiocyanate (FITC), (6) peridinin chlorophyl protein/cyanine 5.5 (PerCP-Cy5.5), (7) to phycoerythrin (PE), (8) phycoerythrin/cyanine7 (PE-Cy7), (9) allophycocyanine (APC), and (10) allophycocyanine/H7 (APC-H7), APC-C750 or APC-Alexa750.

A suitable label may be directly or indirectly linked to the reagent or ligand via the use of a suitable tag. In a preferred embodiment, the detectable label is linked to streptavidin. Fluorochrome reagents are useful in panel reactivity assays, where a pool of two or more defined reagents or ligands are each conjugated to a different fluorochrome and added to a sample. A number of biomarkers may be tested at one time permitting multiplexing from a single blood draw. A blood sample is taken from a subject suspected of having a hypersensitivity or who has received or who is receiving allergen immunotherapy.

The method of the present invention further comprises contacting the sample with a reagent that allows one or more, preferably two or more, biomarkers to be identified. Typically, molecule is bound or is itself the detectable label. For example, the molecule may be a fluorescent dye, antibody, nucleotide probe or enzyme that leads to substrate being produced. Alternatively, the reagent is linked to a tag that facilitates binding to the detectable label. For example, the tag may bind non-covalently to, or form a covalent interaction with, the detectable label. Suitable tags are known in the art and have been described herein.

For example, the reagent is an antibody that detects a biomarker of interest and the detectable label is a fluorochrome. Suitable fluorochromes are known in the art and have been described herein. Several fluorescent conjugated antibodies directed to different phenotypic biomarkers on B-cells may be added to the sample to facilitate the detection and discrimination of different cell types. Preferably, the sample is contacted with a panel of fluorochrome-conjugated antibodies under conditions suitable for antibody binding to their respective antigens.

The sample may be contacted with all the antibodies simultaneously, i.e. with a cocktail, mixture or composition of antibodies. However, it may be suitable to add the antibodies in two or more steps. For example, a two-step incubation may be performed when both surface membrane and intracellular staining is necessary. In such cases, first the surface membrane staining is performed followed by fixation and permeabilization to facilitate cytoplasmic staining. In each case, unlabelled antibodies may be used however; multiple incubations and wash steps may be required. Preferably, complex staining is not generally preferred in routine diagnostic testing.

Any suitable phenotypic biomarker can be used to identify a cell of interest. Suitable B cell biomarkers are CD19, CD20, CD79a or CD22. Most preferably, the CD19 antigen.

It may be useful to include in the panel of fluorochrome-conjugated antibodies to further characterise the immune cells of interest, most preferably B cells. For example, the antibody is reactive with a biomarker for characterisation of Bmem or plasma cells, preferably a biomarker selected from the group consisting of CD23, CD27, CD38, CD40, CD80, CD86, CD148, CD180, TACI, CD200, CD73 and CD62L.

Allergen specific B-cells, such as allergen specific Bmem, may be identified using a labelled allergen (for example a fluorescently labelled allergen). A fluorescently labelled allergen for detection of allergen-specific Bmem is likely to provide the highest sensitive and specificity for detecting the presence of, or any changes to the percentage or number of, allergen-specific Bmem.

### Samples

Any biological sample that is known or suspected to contain a B-cell, preferably Bmem, is contemplated for use in the invention.

The term sample, as used herein, shall include blood samples but may also include hematopoietic biological samples such as lymph, leukopoiesis product, bone marrow and the like; also included in the term are derivatives and fractions of such fluids. The blood sample is drawn from any site for example by venepuncture. Blood samples will usually be from about 1 to 100 ml of whole blood, i.e. from 105 to 107 nucleated blood cells, and may be treated with anticoagulants, e.g. heparin, EDTA, citrate, acid citrate dextrose or citrate phosphate dextrose, as known in the art.

The sample may be a bodily fluid, for example a blood sample as discussed above. Alternatively, the same may be a tissue sample. The sample may contain a bodily fluid and a tissue sample.

The blood sample may be a whole blood, buffy coat, peripheral blood mononuclear cell (PBMC), cord blood, purified or sorted cell population or bodily fluid. Bodily fluids include lymph, semen, nasal secretions, bronchial secretions, alveolar fluid, cerebrospinal fluid, endolymph, synovial fluid, pleural fluid, pericardial fluid (pericardial liquor), menstrual fluid, or combinations thereof.

The tissue sample may be selected from tonsil, lymph node, bronchial, nasal or gut or skin biopsy. Preferably, the tissue sample is treated to form a single cell suspension. Forming a single cell suspension may be through a mesh filter for tonsil, thymus or lymph node. Alternatively, forming a single cell suspension may be via tissue digestion and then using a mesh through filter.

Specifically, in relation to biological samples known or suspected to contain B cells, the sample may be blood, bone marrow or lymphoid tissue. The tissue may be selected from tonsil, lymph node, bronchial, nasal or gut biopsy. Alternatively, the blood sample may be a whole blood sample, buffy coat sample, peripheral blood mononuclear cell (PBMC) sample, cord blood, purified or sorted cell population or bodily fluid. Bodily fluids include samples from the group consisting of lymph amniotic fluid, nasal secretions, bronchial secretions, alveolar fluid, endolymph, pericardial fluid (pericardial liquor), peritoneal fluid, breast milk, or combinations thereof.

The sample may be taken from any mammal including primate. In particular, human, murine, more particularly mouse, equine, bovine, ovine, porcine, canine, feline etc. Whole blood can be draft from the sample using any acceptable procedure. The use of whole blood allows detection of effector cells such as eosinophils and basophils. Alternatively, the blood samples may be resuspended in a solution that selectively lyses erythrocytes, e.g. ammonium chloride-potassium; ammonium oxalate, etc.

### Detection Methods

The detection of reagents or ligands linked to detectable labels binding to biomarkers on B cells may be performed by flow cytometry or microscopy. These methods are practiced as known in the art. The use of flow cytometry or microscopy may be used in conjugation with other cell phenotyping agents.

Where the sample is bound to a fluorochrome selective reagent is used, flow cytometry or microscopy may be used to detect the presence of B-cell labelled with the reagent or ligand. Such methods are practiced as known in the art. biomarker

For example, the sample is subjected to multi-color flow cytometry and gated for B-cells based on the forward scatter and side scatter, typically followed by exclusion of cell doublets and multiplets in an e.g. forward scatter-pulse area versus forward scatter pulse-height bivariate dot plot, according to conventional criteria.

### Monitoring Allergy Treatment Success

Methods of the present invention include monitoring or determining the success of allergen immunotherapy. For instance, the monitoring of treatment efficacy of anti-IgE therapy or allergen immunotherapy (oral/subcutaneous). The allergen immunotherapy may be allergen specific or non-allergen specific (e.g. omalizumab).

In particular, the invention relates to monitoring of the efficacy of allergen immunotherapy. Allergen immunotherapy (also termed hyposensitization therapy, immunologic desensitization, hyposensibilization, or allergen immunotherapy) includes immunotherapy for allergic disorders in which the patient is vaccinated with increasingly larger doses of an allergen with the aim of inducing immunologic tolerance. Allergen specific immunotherapy is the only treatment strategy which treats the underlying cause of the allergic disorder. It can either reduce the need for medication, severity of symptoms or eliminate hypersensitivity altogether. Allergen can be administered under the tongue (sublingually), by injections under the skin (subcutaneous) or in some instances administered by intradermal injection.

The immune system of allergy affected individuals misinterprets a usually innocuous substance as a disease agent and begins producing IgE. This is called the ‘primary antibody response.’ The IgE produced during this response binds to basophils in the bloodstream and to a similar type of cell called mast cells in the tissues. When the person again encounters the allergen, these basophils and mast cells that have bound to IgE release histamine, prostaglandins, and leukotrienes, which causes inflammation of the surrounding tissues, resulting in allergic symptoms. Immunotherapy via repeated exposure to a specific allergen via either sublingual, subcutaneous intradermal, epicutaneous or intralymphatic route leads to a desensitisation to the allergen and thus a reduction in allergic symptomatology and use of symptomatic based treatments. The exact mechanism is not fully understood but it is accepted that immunotherapy causes modification of the immune system. This modification leads to changes in IgE synthesis and the production of IgE blocking antibodies which thus reduces the immune systems allergic response to specific allergens. There is also a shift from Th2 responses towards regulatory T cells. The molecular mechanism of such immunotherapy can be partly interpreted as that there occurs induction of allergen-specific IgG to neutralize the allergen instead of induction of allergen-specific IgE.

As will be appreciated by the skilled person, a method of the present invention is highly suitable to monitor any quantitative changes in allergen-specific B cells in a biological sample of a patient suffering from an allergy and/or receiving allergen immunotherapy. To that end, the IgG-expressing cells (be it memory B cells and/or plasma cells) are easily identified and quantitated by staining with a reagent or ligand targeting a biomarker of interest, the reagent or ligand being provided with a detectable label like a fluorochrome. Accordingly, provided is a method for the monitoring of treatment efficacy of allergen immunotherapy. The method comprises analyzing memory B cell subsets in a biological sample isolated from a subject receiving said immunotherapy (oral/subcutaneous) using a procedure as described herein above for detecting allergen IgG+ memory B cells.

A positive response to allergen immunotherapy may be any one or more of (where the first sample is prior to immunotherapy and the second is during, or after, immunotherapy):
an increase in the level or amount of one or more biomarkers selected from IGHE, ITGB1, IL13RA1, CD99, FCRL3, FCRL2, SIGLEC10, CD1C, FCER2, IL4R, IgE, CD29, IL13Rα, CD99, FCRL3, FCRL2, SIGLEC10, CD1c, CD23, and IL4Rα in B-cells in the second sample compared to the first sample.
a decrease in the level or amount of one or more biomarkers selected from CD69, IGHD, CXCR4, FCRL3, FCRL2, FCRL5, CD1C, FCER2, IL4R, CD69, IgD, CXCR4, FCRL3, FCRL2, FCRL5, CD1c, CD23, IL4Rα in B-cells in the second sample compared to the first sample
an increase in the level or amount of one or more biomarkers selected from IGHE, PPP1R18, ITGB1, PARM1, IgE, phostensin and CD29 in B-cells in the second sample compared to the first sample
a decrease in the level or amount of one or more biomarkers selected from c-jun and CD69 in B-cells in the second sample compared to the first sample

A positive response to allergen immunotherapy preferably occurs at least 4 months after initiation of treatment of the allergen immunotherapy, for any period of time up to 5 years, or any other period described herein.

### Kits

A kit may be provided for the practice of the subject invention. For example, the kit may include one or a panel of reagents or ligands linked to a detectable label as described herein, and, optionally, other antibodies that phenotype the cells of interest. Optionally, a kit of the invention is packaged with instructions for use in a method described herein.

A still further aspect of the invention relates to a diagnostic kit comprising reagents for performing a method herein disclosed. In one embodiment, it is a diagnostic assay kit to determine efficacy of an allergen immunotherapy.

In another aspect of the present invention, there is provided a kit for determining efficacy of an allergen immunotherapy in a subject comprising a means of detecting changes in the level or amount of transcripts, amplification products and/or proteins, wherein the transcripts, amplification products and/or proteins are selected from the group consisting any one or more of IGHE, ITGB1, CD69, IL13RA1, CD99, IGHD, CXCR4, FCRL3, FCRL2, FCRL5, SIGLEC10, CD1C, FCER2, IL4R or proteins: IgE, CD29, CD69, IL13Rα, CD99, IgD, CXCR4, FCRL3, FCRL2, FCRL5, SIGLEC10, CD1c, CD23, IL4Rα, and optionally PPP1R18, PARM1, phostensin, and c-jun, and wherein the kit may include written instructions.

The kit may further comprise any additional reagent, buffer, or device for use in a method of the invention. For example, it may contain reagents to prepare a standard curve, to calibrate the flow cytometer, positive controls, negative controls, and the like.

### EXAMPLES

The following experimental data describes the following:
Patient cohort with RGP allergy (n=27); 13 patients were treated with 4 months SLIT, which induced an anticipated response with an increased in allergen-specific serum IgE and IgG4 levels; 14 patients untreated and these showed a decline in allergen-specific serum IgE, and no changes in IgG2 and IgG4 levels.
The inventors describe for the first time the effect of SLIT for on allergen-specific memory B cells (Bmem). The inventors have identified distinct Bmem transcriptional profiles and demonstrated using pseudotime analysis that SLIT induces differentiation towards two unique cell fates with distinct transcriptional profiles.
SLIT induced differentiation towards a transcriptional profile associated with IGHE and IGHG4-expressing Bmem (pseudotime cluster 4).
Expression of IL13RA1 and IL4R were increased in cluster 4 as compared to clusters 1 and 3.
Cluster 4 also expressed higher levels of FCER2 (CD23) relative to clusters 1 and 3.
14 surface-expressed proteins as candidate markers for treatment success of allergy
Surface protein expression of IgG4, CD29 and CD23 increased on Lol p 1+ Bmem in RGP patients after 4 mo SLIT, and not in patients who did not receive SLIT in that 4 mo interval
Surface expression of IgG4, CD29 and CD23 increased on Api m 1+ Bmem of bee venom allergic patients at day 63 after start of ultra-rush AIT.
Numbers of CD29+ antigen-specific Bmem at 4 months of SLIT are a strong predictor of treatment success.
Surface protein expression of IL4Rα increased on Lol p 1+ Bmem in RGP patients after 4 mo SLIT.

### Example 1—Materials and Methods

### Study Participants

Subjects with ryegrass pollen (RGP) allergy with moderate to severe seasonal allergic rhinitis (SAR) with or without asthma, and serum RGP-specific IgE of ≥0.35 KUA/L (ImmunoCAP) were recruited from the Allergy Clinics of The Alfred and Box Hill Hospitals, Melbourne, Victoria, Australia (Alfred Ethics Committee project number 514/13). Enrolment into the study was performed after the decision on the treatment plan for the upcoming pollen season was made, and after written informed consent.

All patients were permitted standard care with anti-histamines and local symptom relief including topical intranasal corticosteroids. Subjects who received sublingual allergen immunotherapy (SLIT) were treated with a commercial 5-grass pollen SLIT tablet (Oralair®; Stallergenes, Antony, France) using a 4-month (April/May-September/October) daily regimen completed prior to the Australian grass pollen season in 2019. Treatment with Oralair® involved dissolution under the tongue (at least 2 minutes) followed by swallowing the residue. The treatment regimen comprised: day 1 —1 tablet 100 IR (index of reactivity); day 2-2 tablets 100 IR; day 3 to day 120-1 daily tablet 300 IR. Blood samples from all participants were collected prior to treatment initiation (April/May) and after 4 months of treatment (September/October) prior to the start of the Australian grass pollen season.

Subjects with bee venom (BV) allergy, diagnosed on the basis of a systemic allergic response to a bee sting and serum BV-specific IgE of ≥0.35 KUA/L (ImmunoCAP), were recruited from the Allergy Clinics of The Alfred, Melbourne, Victoria, Australia (Alfred Ethics Committee project numbers 245/16 and 297/13). Enrolment into the study was performed after the decision was made to start ultra-rush immunotherapy, and after written informed consent was obtained.

Participants without RGP allergy or BV allergy were included as controls on the basis of no clinical history of either disease, no detectable specific IgE to RGP and negative basophil activation test (BAT) to RGP extract (Monash University project 2016-0289).

Exclusion criteria for all subjects were immunodeficiency, AIT within the last five years, and treatment with continuous oral corticosteroids and/or β-blockers. The use of symptomatic medications (incl. antihistamines and topical corticosteroids) for allergic rhinitis was permitted. The study was conducted according to the principles of the Declaration of Helsinki, and written informed consent from each participant was obtained prior to inclusion.

### Participant Characteristics

Fifty RGP-allergic patients (18-65 years; 52% female) were included with moderate to severe SAR with or without asthma, and serum RGP-specific IgE of ≥0.35 KUA/L (ImmunoCAP). Twenty-seven participants were sampled at t=0, following which thirteen received SLIT, fourteen received standard-of-care treatment alone and twenty-three declined further research follow up. Seventeen BV-allergic patients were included with a history of an anaphylactic response to a bee sting (median 57 years, range 18-69 years; 29% female) and serum BV-specific IgE of ≥0.35 kU_{A}/L (ImmunoCAP).

### Blood Sampling

Heparinised or EDTA blood samples were processed within 24 hours of collection for PBMC isolation and storage. Serum was isolated concurrently from silica-coated collection tubes. Serum RGP-specific-IgE levels were measured by ImmunoCAP using allergen extracts as per manufacturer's instructions at the Alfred Pathology Services (Alfred Hospital, Melbourne, Australia). Serum IgE specific for Lol p 1 (the major allergen of RGP, Lolium perenne) was measured by a semi-quantitative in-house ELISA as described previously. ^{1, 2}

### Enzyme-Linked Immunosorbent Assay (ELISA)

ELISA plate wells were coated with recombinant monomeric, non-biotinylated Lol p 1 (MyBiosource, San Diego, CA, US), blocked with 5% skim milk powder in PBS and incubated with serial dilutions of serum samples. For Lol p 1-specific IgG₂ and IgG₄ ELISAs, standard curves were generated with chimeric Lol p 1-specific IgG₂ or IgG₄ mAb generated from a previously described mouse hybridoma producing Lol p 1-specific antibody (see next section).³ For Lol 1-specific IgE ELISAs, separate wells were incubated with a range of concentrations of purified recombinant human IgE (clone AbD18705; Bio-Rad, Puchheim, Germany) to generate a standard curve for relative quantification of IgE in serum. Antibody bound to Lol p 1 was detected using polyclonal rabbit anti-hlgE (Agilent, Santa Clara, CA, US), biotinylated anti-hIgG₂ (clone HP6002, Thermo Scientific) or biotinylated anti-hIgG₄ (clone HP6025, Sigma Aldrich, St. Louis, MO, US) followed by polyclonal goat anti-rabbit HRP (Promega, Madison, WI, US). ELISA were developed using TMB (Thermo Fisher Scientific, Waltham, MA, US) before the reaction was stopped with 1M HCl and absorbance measured at OD 450 nm on a Multiskan Microplate Spectrophotometer (Thermo Fisher Scientific). Wells without allergens were used to determine background values that were subtracted from allergen-specific IgE values and results are expressed in arbitrary units (AU).

Production of Recombinant IgG₂, IgG₄, Lol p 1 and Api m 1

Chimeric IgG₂ and IgG₄ mAb specific to Lol p 1 were generated based on the Igh and IgK variable regions from a previously described mouse hybridoma producing Lol p 1-specific antibody.³ PCR on mRNA from mouse hybridomas4, 5 with primers specific for mouse Igh and Ig variable regions yielded products that were cloned into either a pWin98 vector containing human IgG₂ or pBAR981 vector containing human IgG₄ constant regions. Resultant plasmids were transfected into Expi293F cells (Thermo Fisher Scientific, Scoresby, Australia). Anti-Lol p 1 IgG₂ and IgG₄ were purified by Protein A column (GE Healthcare Amersham Biosciences, Uppsala, Sweden) and Lol p 1-specificity confirmed by ELISA (data not shown).

Recombinant Lol p 1 and Api m 1 were generated in Spodoptera frugiperda 21 (Sf21) insect cells as previously described.⁶ Briefly, protein sequences of Api m 1.0101 and Lol p 1.0101 were obtained from the Allergen Nomenclature website (allergen.org).^{7, 8} Both constructs were generated with the N-terminal leader sequence of Api m 1 for secretion, a 6-His tag for purification and a BirA tag for biotinylation. The Api m 1 (H67Q) and Lol p 1 (H104V) mutations were introduced to prevent unwanted effects of catalytic activity.⁹ The Api m 1 and Lol p 1 constructs were codon-optimized for Spodoptera frugiperda and cloned into the pFastBac vector (Thermo Fisher Scientific), prior to incorporation into a Bacmid for baculovirus production. Bacmids were transfected into Sf21 cells, which were subsequently cultured at 27° C. Supernatants from infected Sf21 cultures were clarified by centrifugation and the 6-His tagged proteins were purified through retention on a cobalt column. Supernatants were gravity-fed through a 25 ml column packed with 4 ml Talon NTA-cobalt-agarose beads (Clontech, Mountain View, CA, US). Beads were washed with PBS and proteins were eluted with PBS, pH 8.5, containing 200 mM imidazole. Eluate was dialyzed against 10 mM TRIS, pH 7.5. Purified recombinant proteins were incubated overnight at RT with BirA enzyme for targeted biotinylation (2.5 μg/ml BirA in 10 mM TRIS containing 62.5 mM Bicine-HCl, 12.5 mM ATP, 12.5 mM MgOAc, 62.5 μM D-biotin). Following subsequent dialysis against PBS, tetramerization was performed with fluorochrome-conjugated streptavidin (BV711 and BUV395 conjugates; BD Biosciences, San Jose, CA, US) at a 4:1 molar ratio of Lol p 1: streptavidin.

### Cell Sorting and Flow Cytometry

Isolation of Lol p 1-specific Bmem for single cell transcriptomics was performed using fluorescence-activated cell sorting. Briefly, ten million PBMC were incubated with a 10-color cocktail of 2 Lol p 1 allergen tetramers (APC and PE) and 7 antibodies against B cell markers (CD19, CD38, CD27, IgM, IgD, IgG, IgA,) for 15 minutes at room temperature in 300 μL total volume. Lol p 1-specific memory B cells (Lol p 1+CD19+ CD27+CD38dim and Lol p 1+CD19+ CD27-CD38dimlgM-IgD-IgG+ or Lol p 1+CD19+ CD27-CD38dimlgM-IgD-IgA+) were defined and sorted from 4 patients before and after SLIT on a 6-laser FACSAria™ Fusion (BD Biosciences, Franklin Lakes, NJ, US).

Detailed immunophenotyping of Lol p 1 specific Bmem was performed on 27 paired samples from RGP allergic patients, and Api m 1 specific Bmem on 17 paired samples from BV allergic patients. B cell subsets were defined as described previously.¹⁰⁻¹² Briefly, within the CD19+ B-cell population, the proportions of IgM+ Bmem (CD38dimCD27+ IgM+), IgG+ Bmem (CD38dimlgD-IgM-IgG+) and IgA+B mem (CD38dimlgD-IgM-IgA+) were determined. Furthermore, allergen-specific Bmem were quantified and evaluated for expression of Ig isotypes and surface markers.

Data were acquired on a 5-laser BD LSRFortessa X-20 (RGP-allergic) and a 5-laser Cytek Aurora with instrument set-up and calibration performed using standardized EuroFlow SOPs as described in detail previously.^{32, 33} All flow cytometry data were analyzed with FlowJo v10 software (FlowJo LLC, Ashland, OR, US).

### Single Cell Transcriptome Profiling

FACS purified Lol p 1+ Bmem were labelled with sample tags according to manufacturer's instructions (Human Single-Cell Multiplexing Kit, BD Biosciences). Single-cell capture, cDNA synthesis and library preparation were performed using the BD Rhapsody Single-Cell Analysis System (BD Biosciences) for Whole Transcriptome Analysis, according to manufacturer's instructions. Libraries were sequenced on a single run using an Illumina MiSeq platform (Genomics Hub, The Walter and Elisa Hall Institute of Medical Research, Melbourne, Australia). Raw sequences were processed using the SevenBridges platform (SevenBridges, Boston, MA, US) to identify sample tags, cell barcodes and the number of transcripts per cell. Cells without identifiable sample tags were excluded from further analysis. Single cell transcriptomics data were analyzed with SeqGeq v1.6 software package (FlowJo LLC). Read counts were normalized to a total library size of 10,000 reads per cell. Dimensionality reduction by principal component analysis (PCA) determined 25 principal components derived from the top 200 genes with most dispersed gene expression. T-distributed stochastic neighbor embedding (tSNE) analysis was performed with 25 principal components from PCA. Pseudotime analysis to construct single cell trajectories was determined with Monocle v.² plugin. Data dimensionality was reduced by reversed graph embedding with a gene expression filter of one and a cell expression filter of ten.

### Statistics

Differential gene expression from the combined transcripts of all cells before vs. after SLIT was illustrated by volcano plots and significance determined by Bonferroni corrected Mann-Whitney t-test. Genes with a Q-value <0.05 and absolute fold change ≥ 1.5 were considered significantly and relevantly different between the analyzed subsets. Differences in proportions of IGHC transcripts were statistically analyzed with the x² test.

Statistical analyses of flowcytometric data were performed using Graphpad Prism (v8.4.1): paired data were analyzed with the non-parametric Wilcoxon signed rank test and unpaired data with the non-parametric Mann-Whitney U-test. For all tests, p<0.05 was considered significant.

### Example 2—Four-Month SLIT Increases Serum Allergen-Specific IgE and IgG4 Levels

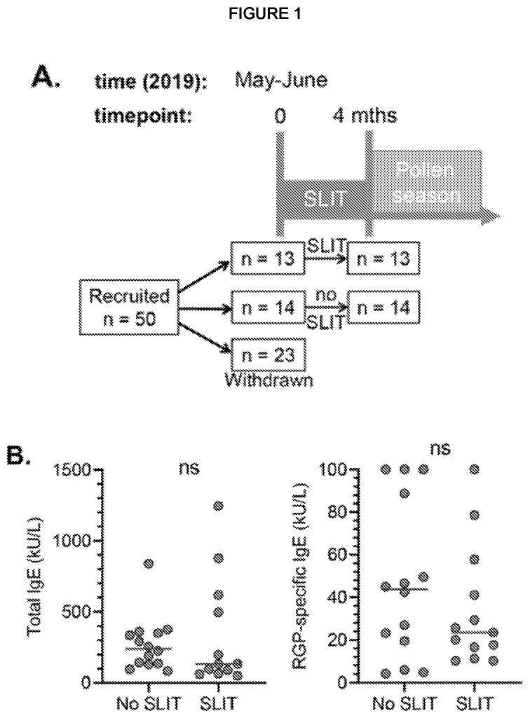

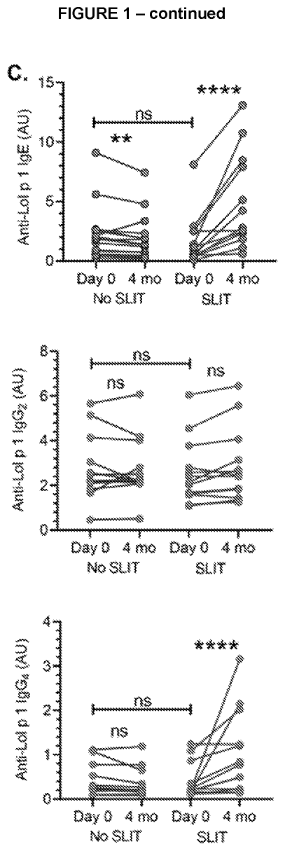

Meta-analyses and clinical trials have confirmed the clinical efficacy of SLIT with a 5-grass mix. Of the 13 patients in this active study arm accessible two years later, 12 (92%) reported clinical benefit, thereby validating the efficacy of SLIT in this cohort. There were no significant differences in total, RGP-specific or Lol p 1-specific IgE at the time of recruitment between patients recruited to receive SLIT or that did not receive SLIT (FIG. 1 B-C). Four months of SLIT increased Lol p 1-specific IgE and IgG₄ (FIG. 1C). In patients that did not receive SLIT, the intervening 4 months saw a slight but significant decrease in Lol p 1-specific IgE, whereas Lol p 1-specific IgG₂ and IgG₄ levels were unchanged (FIG. 1C).

### Example 3—Increased Expression of IGHE and IGHG4 Transcripts in Lol p 1-Specific Bmem after SLIT

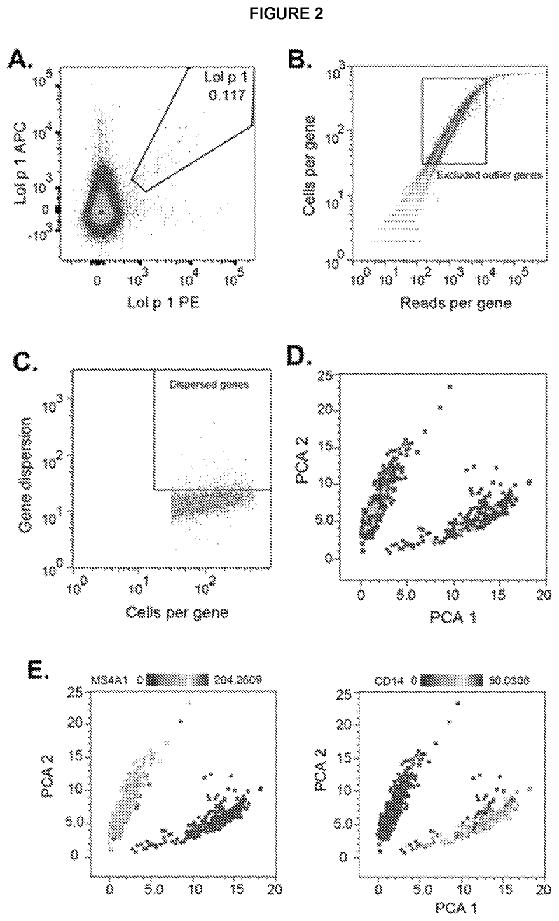

To investigate the immunological effects of SLIT, the inventors conducted single-cell transcriptomics on Lol p 1-specific Bmem from 4 patients before and after 4 months of SLIT. Following electronic gating of CD19+ B cells and CD3+ T cells, Lol p 1-specific B cells were identified by flow cytometry using double discrimination, i.e. double-positivity for two fluorescent Lol p 1 tetramer conjugates (FIG. 2A). Lol p 1 tetramers did not bind to CD3+ T cells (data not shown), nor did B cells bind to the streptavidin-fluorochrome conjugates used to generate these tetramers (data not shown), confirming that the double-positive population constitutes bona fide Lol p 1-specific B cells. A total of 189 cells pre-SLIT and 323 cells post-SLIT were subjected to scRNAseq. Outliers for total reads per gene (<150, >15,000) and cells per gene (<30, >650) were excluded, followed by dimensionality reduction with principal component analysis (PCA) based on the 200 most dispersed genes (FIG. 2B-D). This resulted in 2 main clusters, one expressing canonical B cell genes MS4A1 (CD20) whereas the other was enriched for myeloid gene CD14 (FIG. 2E). The latter population was deemed to consist of monocytes and was excluded, leaving a total of 512 B cells for further analysis.

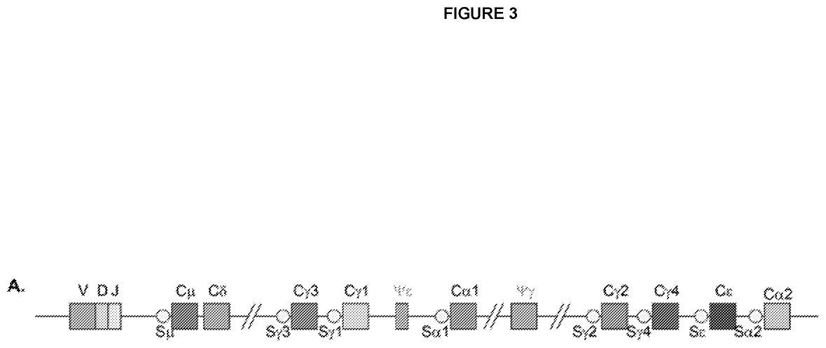

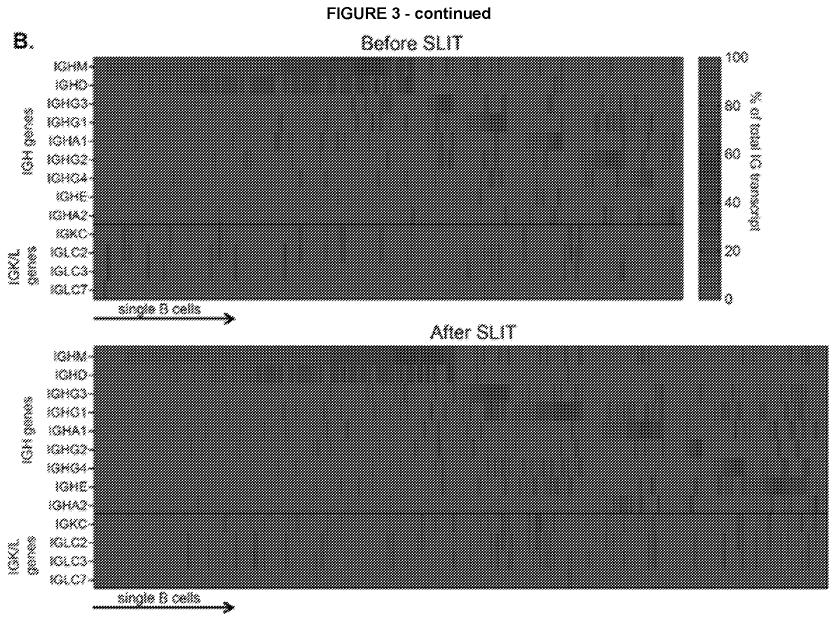

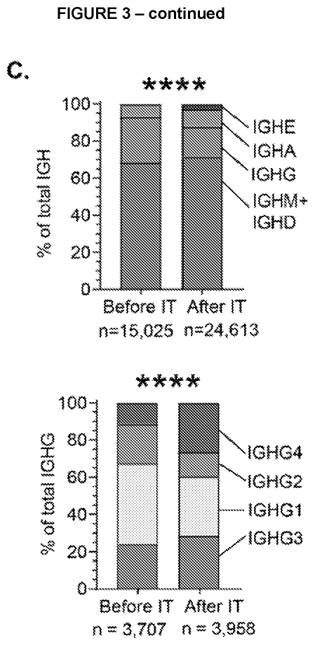

To investigate the Ig gene usage of these Bmem, the inventors assessed the expression of the IGH constant regions (FIG. 3A), as well as expression of of the IGK and IGL light chains. Each cell expressed one predominant IGH isotype and a single IGK or IGL constant region (FIG. 3B). The proportions of IGHE and IGHG4 amongst total IGH transcripts were significantly increased in Lol p 1-specific Bmem after SLIT (FIG. 3C). There were no differences in the proportion of IgK or lgL expression in Lol p 1-specific Bmem before and after SLIT (data not shown). Thus, after 4 months SLIT, both IgE and IgG₄ serum levels as well as B cells expressing IGHE and IGH4 were increased (FIG. 1C).

### Example 4—SLIT Promotes Differentiation of Lol p 1-Specific Bmem into Two Pseudotemporally Distinct Transcriptional Profiles

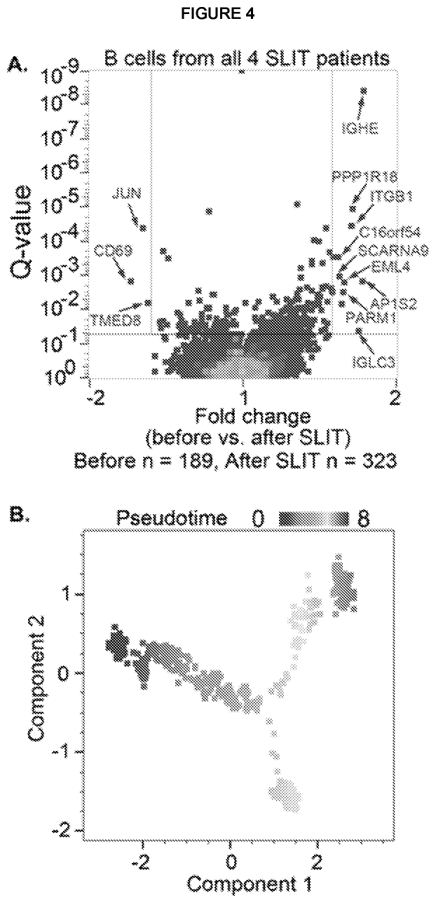

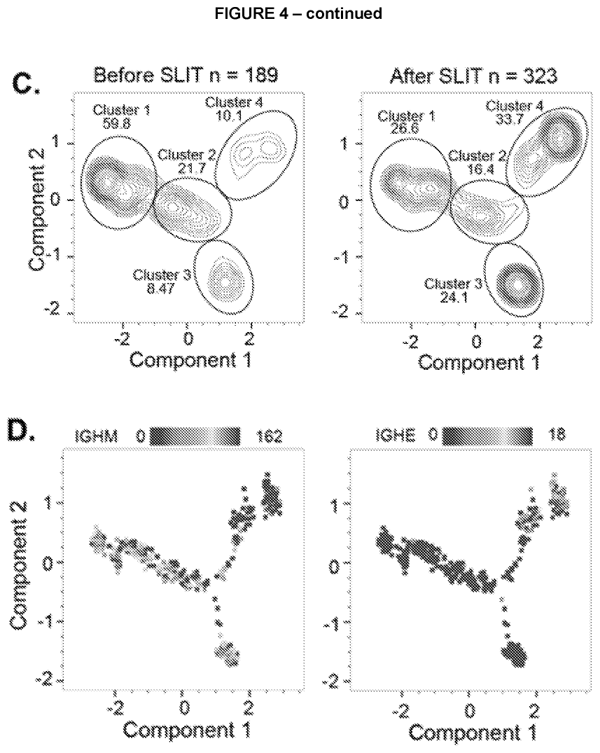

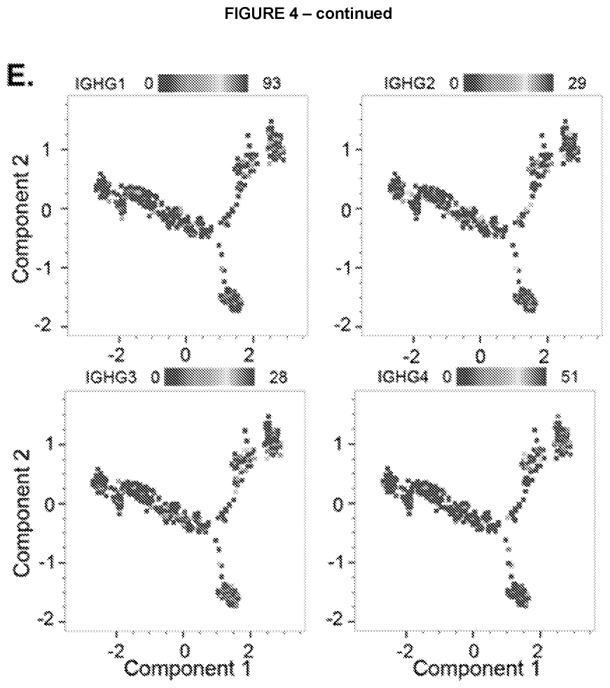

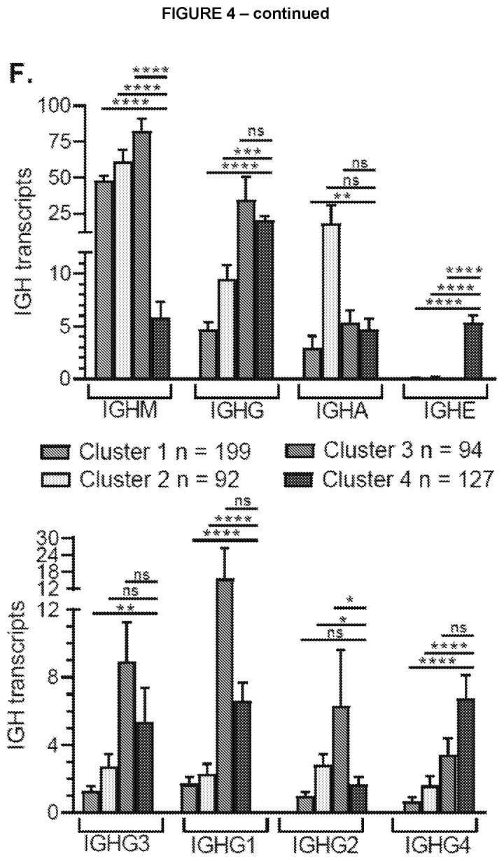

To further investigate the transcriptional changes of SLIT on Bmem, 15 relevant differentially-expressed genes were identified from Lol p 1-specific Bmem before and after SLIT on the basis of a fold change >1.5 and Q<0.05 (Table 3). Of these 15 genes, 12 were increased in expression level after SLIT and 3 were decreased. Only 3 genes were considered relevant biomarkers (IGHE, ITGB1, and CD69), as other genes were either not surface-expressed, or were the result of donor mix, which included Ig lambda light chain (IGLC3) and HLA (HLA-DRB5). Most notably, SLIT increased expression of IGHE (IgE), phosphatase 1 F-actin cytoskeleton targeting unit PPP1R18 (phostensin), beta 1 integrin ITGB1 (CD29) involved in lymphocyte tissue homing and oncogene PARM1 which is associated with CD8+ T cell leukemia and IgG+ memory B cells of a transitional memory B cell state (FIG. 4A). In contrast, SLIT reduced overall expression of oncogenic transcription factor JUN (c-jun) and C-type lectin CD69, the latter having been associated with preventing egress of B cells from lymphoid organs.

Numerous differentially-expressed genes with <1.5 fold change after SLIT suggested that SLIT-induced changes to the transcriptional profile of Lol p 1-specific Bmem may have been taking place in transcriptionally distinct clusters of B cells that cannot be identified by pre vs post SLIT comparison alone. Using these genes to generate a PCA and tSNE analysis did not yield distinct clustering of Bmem before and after SLIT suggesting. To identify clusters of transcriptional profiles associated with changes induced by SLIT, the inventors conducted a pseudotime analysis including all differentially-expressed genes with a significant change (Q<0.05) irrespective of the fold-change (n=272 genes). The B cell cluster expressing IGHM and IGHD was designated pseudotime zero due to known class-switching from IgM+B cell precursors. Clustering of total Lol p 1-specific B cells using this pseudotime analysis pathway revealed two distinct branches of B-cell differentiation (FIG. 4B). Four distinct B cell clusters could be identified along this differentiation pathway (FIG. 4C). SLIT decreased Bmem in the least differentiated state of cluster 1 (pseudotime=0-2) and drove Bmem toward the most differentiated states in cluster 3 (pseudotime=5-6) and cluster 4 (pseudotime=6-8) (FIG. 4C). Heatmaps of IGH gene expression demonstrated that an increase in pseudotime was associated with a shift from IGHM to expression of Ig class-switched isotypes, particularly IGHE in cluster 4 (FIG. 4D, 4E). Indeed, Bmem in cluster 4 were enriched for IGHE, IGHG1 and IGHG4 expression whereas cells in cluster 3 predominantly expressed IGHM, IGHG3 and IGHG1 (FIG. 4F). SLIT increased the proportion of B cells in clusters 3 and 4, suggesting that SLIT induced Ig class-switching and differentiation of Ag-specific Bmem towards either of these two distinct transcriptional profiles.

### Example 5—Gene Expression Associated with Transcriptional Profiles Induced by SLIT

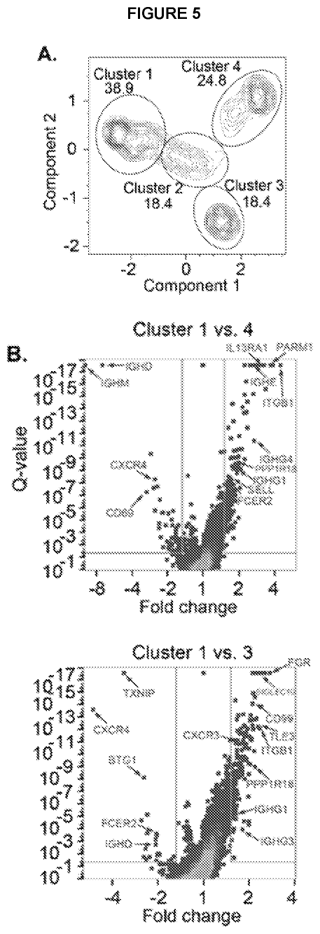

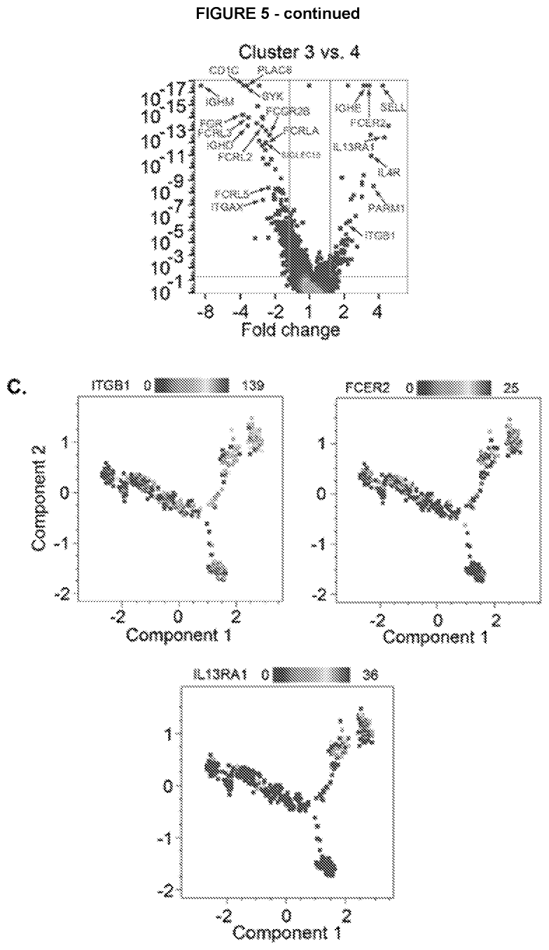

Differentially expressed gene analysis of distinct pseudotime clusters confirmed increases in IGHE, IGHG4 and IGHG1 in cluster 4 compared to cluster 1, whereas cluster 1 was enriched for IGHM and IGHD (FIG. 5A, B). Compared to cluster 1, cluster 4 also had increased expression of immune-related genes such as PARM1, IL13RA1, ITGB1, PPP1R18, SELL, FCER2 and decreased expression of CXCR4 and CD69 (FIG. 5B). Similarly, cluster 3 also had increased expression of ITGB1 and PPP1R18 alongside decreased CXCR4 expression compared to cluster 1 (FIG. 5B). Furthermore, cluster 3 exhibited increased expression of FGR, SIGLEC10, CD99, TLE3 and decreased expression of TXNIP, BTG1 and FCER2 compared to cluster 1. Comparing clusters 3 and 4, cluster 4 had increased expression of SELL, FCER2, IGHE, IL13RA1, IL4R, PARM1 and ITGB1 whereas cluster 3 had increased expression of IGHM, IGHD, CD1C, PLAC8, SYK, FGR, FCGR2B, FCRLA, FCRL2, FCRL3, FCRL5 and ITGAX (FIG. 5B). Due to unique expression patterns of ITGB1, FCER2 and IL13RA1 in pseudotime cluster 4 compared to clusters 1-3, as well as the association of this cluster with IgE and IgG₄ which the inventors have already observed to be increased by SLIT, the inventors wished to visualize the expression of these genes relative to their space in pseudotime to further characterize these cells (FIG. 5C). Expression of these genes changed as pseudotime progressed reflecting the changes in Bmem transcriptional profiles along a SLIT-induced differentiation pathway.

To identify potential biomarkers for early effects of immunotherapy, a selection was made for genes that encoded surface-expressed proteins that were significantly different with a relevant change in at least one of three comparisons:
Pre-vs post-SLIT with a fold change >1.5 fold up or down
Cluster 1 vs cluster 3 with a fold change >2-fold up or down
Cluster 1 vs cluster 4 with a fold change >2-fold up or down

This analysis yielded 14 transcripts with 2 of these meeting all 3 criteria, 6 meeting 2 criteria and the remaining 6 being significantly and relevantly different in one comparison (Table 2).

### Example 6—Confirmation of Biomarkers by Flowcytometry

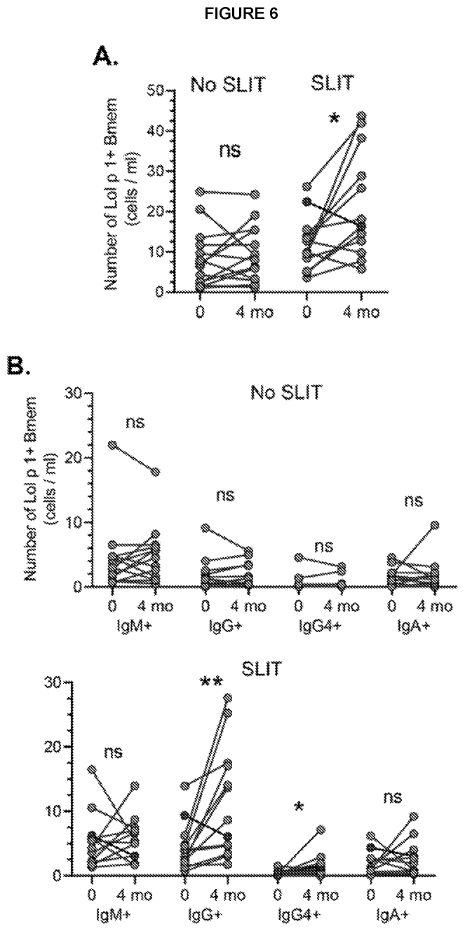

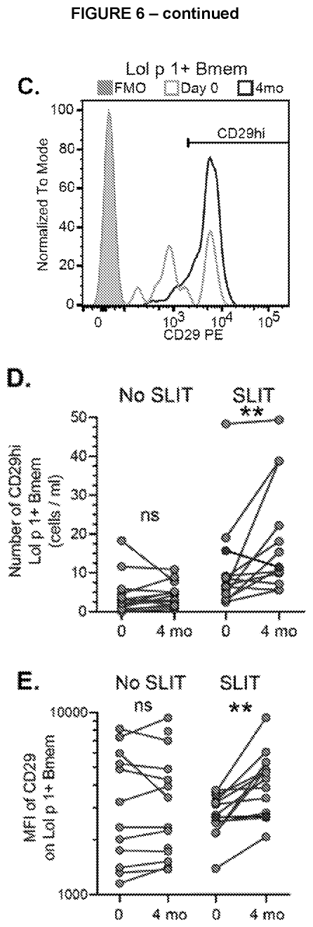

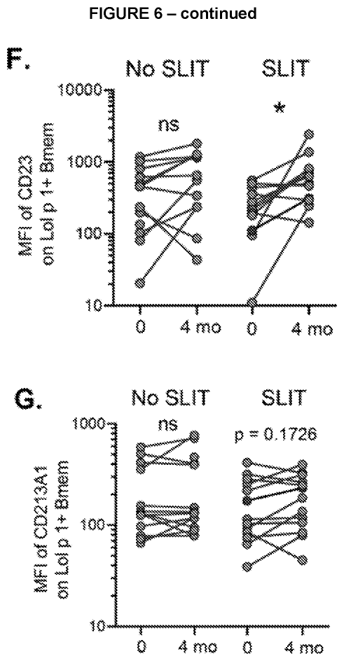

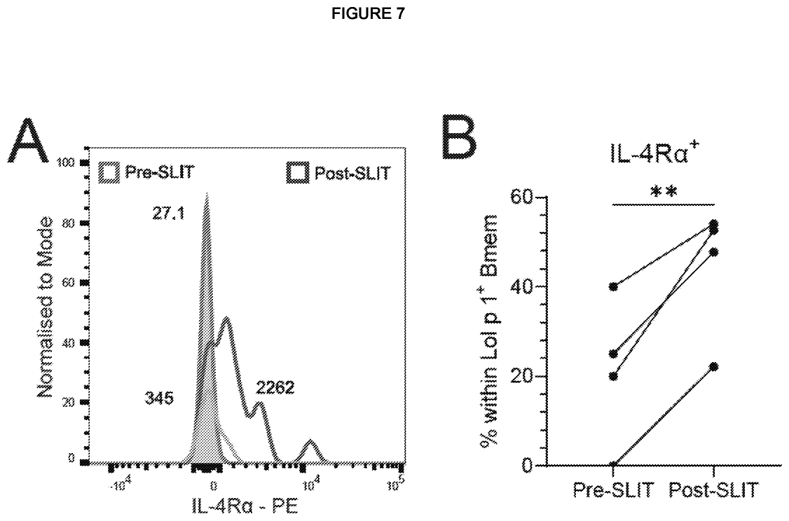

To validate SLIT-induced changes in gene expression identified by transcriptomics, the inventors assessed the cell surface expression of Ig isotypes, CD29, CD23, CD213A1 and IL4Rα Lol p 1-specific Bmem by flow cytometry at timepoints 0 and 4 months from patients that received or did not receive SLIT. In patients that received SLIT, the number of Lol p 1-specific Bmem was increased (FIG. 6A). Both proportion and number of IgG+ (all IgG isotypes) and more specifically IgG₄+ Lol p 1-specific Bmem increased after SLIT, whereas IgM+ Lol p 1-specific Bmem decreased (FIG. 6B). Subjects that did not receive SLIT had no observable change in proportion or number of Lol p 1-specific Bmem over the 4-month time course (FIG. 6A, B). SLIT also increased the proportion and number of CD29hi Lol p 1-specific Bmem and was further associated with increases in CD29, CD23 and IL4Rα staining on Lol p 1-specific Bmem (FIG. 6C-F and FIG. 7). These flow cytometry data correspond with our transcriptomic data that identified increased proportions of Lol p 1-specific Bmem expressing FCER2 (CD23), ITGB1 (CD29) and IL4R (IL4Rα) after SLIT (FIG. 5). In contrast, the inventors did not observe any change in CD213A1 staining on Lol p1-specific Bmem from patients that received SLIT (FIG. 6G). Staining of CD29, CD23 and CD213A1 on Lol p 1-specific Bmem from patients that did not receive SLIT was unchanged (FIG. 6D-G).

As only one subject did not report a benefit of SLIT, the inventors wished to investigate if the numbers and expression profiles of their Lol p 1-specific Bmem were consistent with those patients that had reported a benefit of SLIT (non-responder indicated by red dots and black lines; FIG. 6). This subject exhibited the largest decrease in numbers of total, IgG+ and CD29hi Lol p 1-specific Bmem amongst those patients treated with SLIT (FIG. 6A, B, D). Furthermore, MFI of CD29 on Lol p 1-specific Bmem from this non-responder was unchanged after SLIT (FIG. 6E). Although only an isolated case, this finding suggests that an increase in CD29hi class-switched Lol p 1-specific Bmem may be indicative of successful immunotherapy for grass pollen allergy.

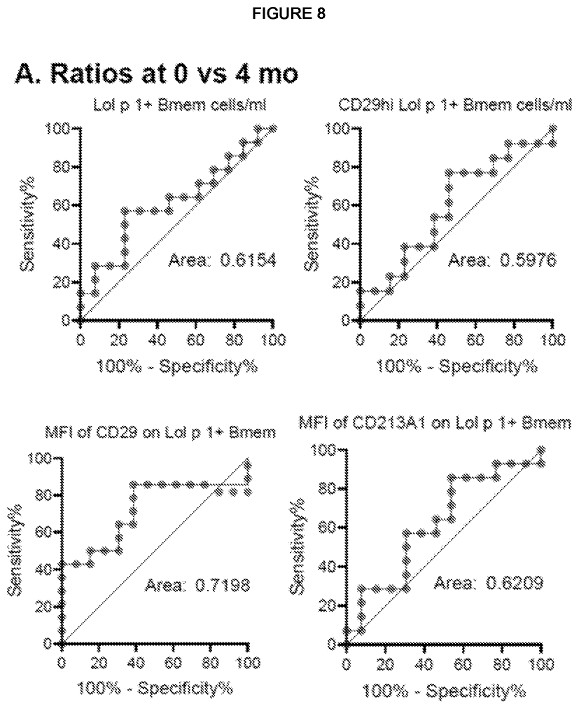

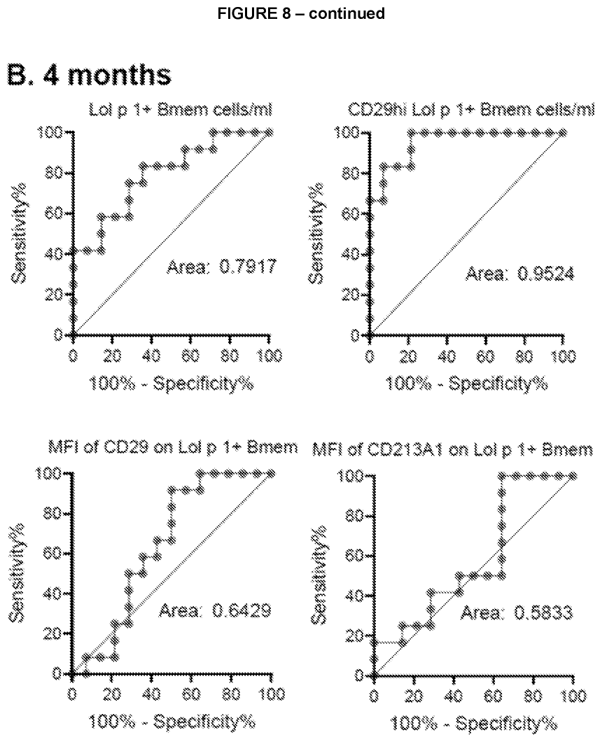

To evaluate the potential of each marker as predictor of treatment outcome, receiver-operator curves were generated for treated vs untreated patients. To evaluate the effects of 4 months, the first evaluation was the ratio of markers after 4 months divided by the pre-treatment values (FIG. 8A) for total Lol p 1+ Bmem, CD29+ Lol p 1+ Bmem, and for expression levels of CD29 and CD213A1 on Lol p 1+ Bmem. CD29 expression levels on Lol p 1+ Bmem was most predictive with an AUC of just below 0.72 (FIG. 8A). Subsequently, the inventors evaluated the markers at 4 months. These yielded higher AUC for Lol p 1+ Bmem (0.79) and CD29+ Lol p 1+ Bmem (0.95), while the AUC for expression levels of CD29 and CD213A1 did not improve (FIG. 8B). Thus, at 4 months of treatment, CD29+ Lol p 1+ Bmem numbers are a very strong indicator of treatment success and more specific than total Lol p 1+ Bmem.

### Example 7—Evaluation of Biomarkers Following Ultra-Rush AIT for Bee Venom Allergy

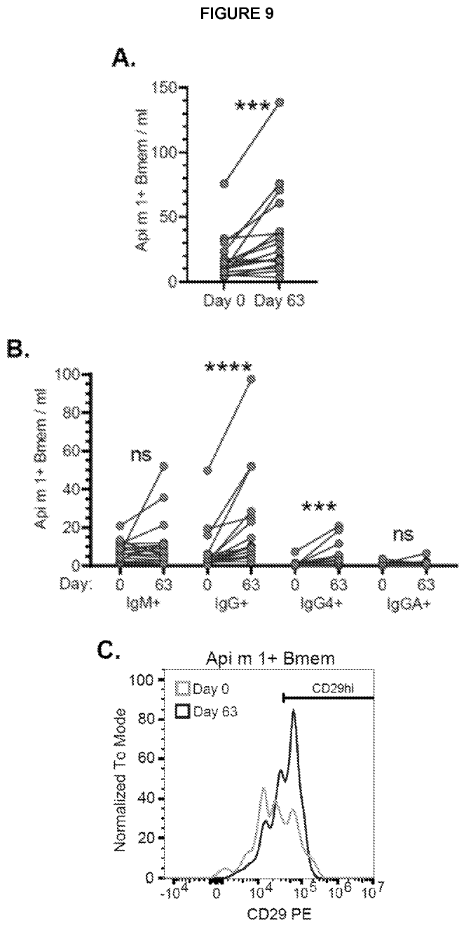

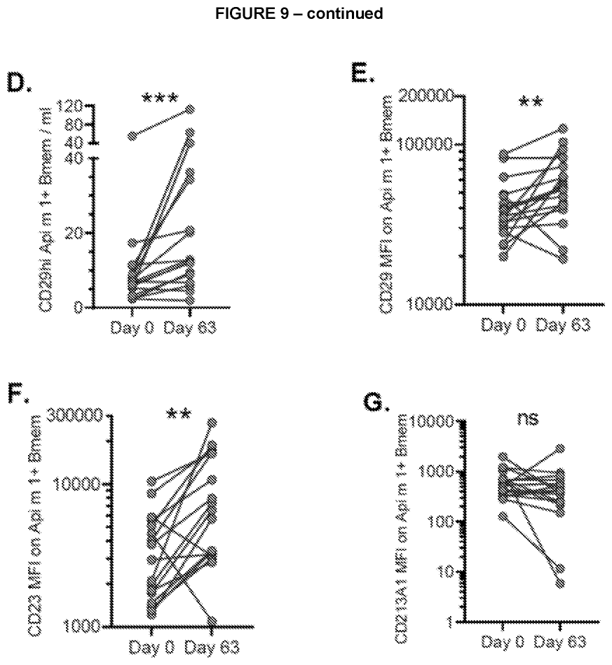

To determine whether the changes in allergen-specific Bmem induced by SLIT for RGP allergy are informative for other allergic conditions as well, these were evaluated on Api m 1-specific Bmem from bee venom allergic patients before and 63 days after start of ultra-rush AIT. Similar to SLIT for RGP, absolute numbers of allergen-specific Bmem (Api m 1+) were increased at day 63 (FIG. 9A). This was predominantly the result of IgG-expressing Bmem and included an expansion of the IgG₄+ subset (FIG. 9B). In addition, day 63 of ultra-rush AIT, the absolute numbers of Api m 1+ Bmem that highly expressed CD29 were significantly increased (FIG. 9 C, D). Furthermore, the expression levels of both CD29 and CD23 on Api m 1+ Bmem were significantly increased after AIT, whereas CD213A1 was not altered (FIG. 9 E-G).

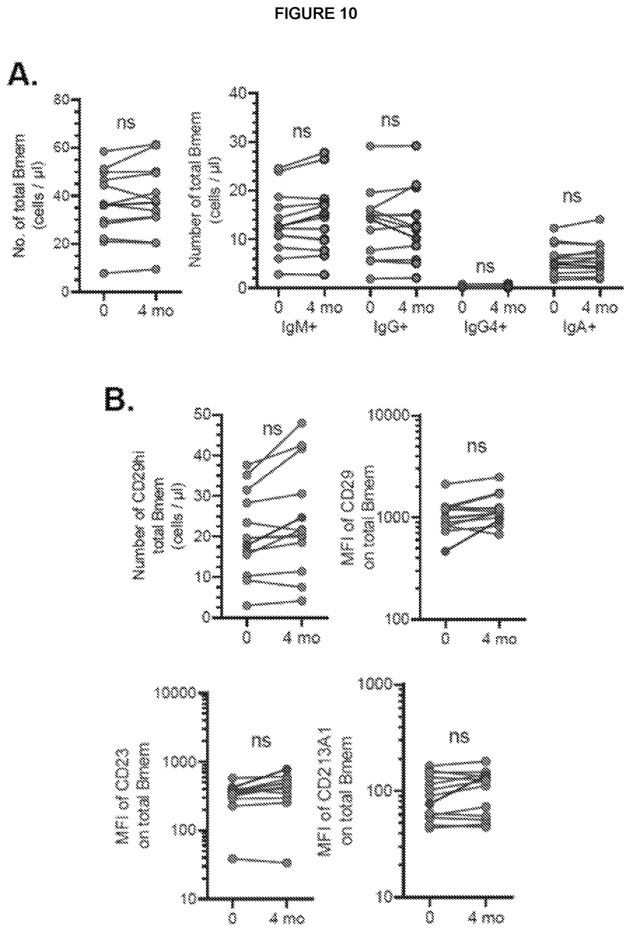

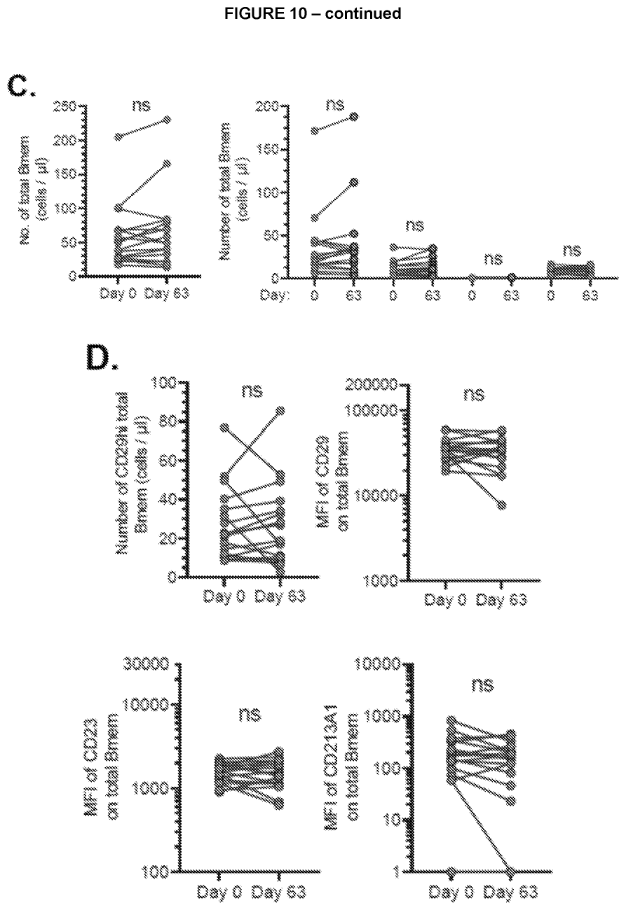

Expression levels of CD29, CD23 and CD213A1 did not alter on total B cells after SLIT for RGP allergy, nor after ultra-rush AIT for bee venom allergy (FIG. 10), strengthening the observations that the effects are antigen-specific.

Thus, single-cell transcriptomics of allergen-specific B cells before and after AIT is a highly sensitive approach for the identification of immunophenotypic changes, which could serve as early biomarkers for prediction of therapy success.

TABLE 2
Differentially-expressed genes encoding surface-expressed proteins

| | | | Fold change | | |
|---|---|---|---|---|---|
| | | | Pre vs | Cluster | Cluster |
| Number | Gene | Protein | Post | 1 vs 4 | 1 vs 3 |
| 1 | IGHE | IgE | 1.73 | 3.00 | — |
| 2 | ITGB1 | CD29 | 1.64 | 4.51 | 2.15 |
| 3 | CD69 | CD69 | 0.61 | 0.34 | 0.41 |
| 4 | IL13RA1 | IL13Rα | — | 3.14 | — |
| 5 | CD99 | CD99 | 1.42 | 2.82 | 2.10 |
| 6 | IGHD | IgD | — | 0.14 | 0.48 |
| 7 | CXCR4 | CXCR4 | — | 0.39 | 0.19 |
| 8 | FCRL3 | FCRL3 | — | 0.40 | 1.34 |
| 9 | FCRL2 | FCRL2 | — | 0.42 | 1.10 |
| 10 | FCRL5 | FCRL5 | — | 0.43 | 0.96 |
| 11 | SIGLEC10 | SIGLEC10 | 1.24 | — | 2.35 |
| 12 | CD1C | CD1c | — | 0.57 | 2.10 |
| 13 | FCER2 | CD23 | 1.37 | 1.62 | 0.49 |
| 14 | IL4R | IL4Rα | 1.28 | 1.44 | 0.42 |

Genes were selected on the basis of encoding surface-expressed proteins, having Q < 0.05 for at least one of the three shown comparisons and having a relevant fold-increase (>1.5 pre vs post and/or >2 between clusters); Numbers are only shown for significant changes (Q < 0.05). Bold font: fold change above threshold.

TABLE 3
Differentially-expressed genes identified from Lol p
1-specific Bmem before and after (pre vs post) SLIT

| | | Pre vs post SLIT | Surface |
|---|---|---|---|
| Number | Gene | fold change | expressed |
| 1 | IGHE | 1.73309 | + |
| 2 | AP1S2 | 1.7213 | − |
| 3 | IGLC3 | 1.69307 | + |
| 4 | PPP1R18 | 1.64833 | − |
| 5 | ITGB1 | 1.63827 | + |
| 6 | EML4 | 1.58806 | − |
| 7 | PARM1 | 1.57863 | − |
| 8 | RAB11A | 1.57398 | − |
| 9 | SCARNA9 | 1.55173 | − |
| 10 | C16orf54 | 1.54218 | − |
| 11 | HLA-DRB5 | 1.50938 | + |
| 12 | MAPK1IP1L | 1.50749 | − |
| 13 | CD69 | 0.605903 | + |
| 14 | JUN | 0.639837 | − |
| 15 | TMED8 | 0.654187 | − |

Genes were selected on the basis of having Q < 0.05 and having a relevant fold-increase (>1.5 pre vs post SLIT). Use of the symbol “+” indicates positive detection of the protein on the cell surface of Lol p1-specific Bmem. Use of the symbol “−” indicates the absence of the protein on the cell surface of Lol p1-specific Bmem.

### REFERENCES

1. Whitrow, M. J., Moore, V. M., Rumbold, A. R., Davies, M. J. Effect of supplemental folic acid in pregnancy on childhood asthma: a prospective birth cohort study. American journal of epidemiology 170, 1486-1493 (2009).
2. Heeringa, J. J., et al. Induction of IgG2 and IgG4 B-cell memory following sublingual immunotherapy for ryegrass pollen allergy. Allergy 75, 1121-1132 (2020).
3. Smart, I. J., Heddle, R. J., Zola, H., Bradley, J. Development of monoclonal mouse antibodies specific for allergenic components in ryegrass (Lolium perenne) pollen. Int Arch Allergy Appl Immunol 72, 243-248 (1983).
4. Seidl, K. J., et al. Frequent occurrence of identical heavy and light chain Ig rearrangements. Int Immunol 9, 689-702 (1997).
5. Tiller, T., Busse, C. E., Wardemann, H. Cloning and expression of murine Ig genes from single B cells. J Immunol Methods 350, 183-193 (2009).
6. Mckenzie, C. I., et al. CytoBas: Precision component-resolved diagnostics for allergy using flow cytometric staining of basophils with recombinant allergen tetramers. Allergy, 76, (10) 3028-3040 (2021).
7. Chan, S. K., et al. Keeping Allergen Names Clear and Defined. Front Immunol 10, 2600 (2019).
8. Pomes, A., et al. WHO/IUIS Allergen Nomenclature: Providing a common language. Mol Immunol 100, 3-13 (2018).
9. Grobe, K., Poppelmann, M., Becker, W.-M., Petersen, A. Properties of group I allergens from grass pollen and their relation to cathepsin B, a member of the C1 family of cysteine proteinases. European Journal of Biochemistry 269, 2083-2092 (2002).
10. Berkowska, M. A., et al. Human IgE (+) B cells are derived from T cell-dependent and T cell-independent pathways. J Allergy Clin Immunol 134, 688-697.e686 (2014).
11. de Jong, B. G., et al. Human IgG2- and IgG4-expressing memory B cells display enhanced molecular and phenotypic signs of maturity and accumulate with age. Immunol Cell Biol 95, 744-752 (2017).
12. Heeringa, J. J., et al. Expansion of blood IgG (4) (+) B, T (H) 2, and regulatory T cells in patients with IgG (4)-related disease. J Allergy Clin Immunol 141, 1831-1843.e1810 (2018).

1. A method of determining efficacy of an allergen immunotherapy in a subject, the method comprising:
providing a first sample obtained from a subject before receiving allergen immunotherapy;
providing a second sample obtained from the subject who has received, or who is receiving, allergen immunotherapy;
wherein the first and second samples comprise allergen specific Bmem; and
determining the proportion or number of CD29+ allergen specific Bmem or CD29hi allergen specific Bmem in the first and second samples;
wherein an increase in the proportion or number of CD29+ allergen specific Bmem or CD29hi allergen specific Bmem in the second sample compared to the first sample indicates efficacy of an allergen immunotherapy in a subject.

2. A method of determining efficacy of an allergen immunotherapy in a subject, the method comprising:
providing a first sample obtained from a subject before receiving allergen immunotherapy;
providing a second sample obtained from the subject who has received, or who is receiving, allergen immunotherapy;
wherein the first and second samples comprise allergen specific Bmem; and
determining the proportion or number of CD23+ allergen specific Bmem or CD23hi allergen specific Bmem in the first and second samples;
wherein an increase in the proportion or number of CD23+ allergen specific Bmem or CD23hi allergen specific Bmem in the second sample compared to the first sample indicates efficacy of an allergen immunotherapy in a subject.

3. A method of determining efficacy of an allergen immunotherapy in a subject, the method comprising:
providing a first sample obtained from a subject before receiving allergen immunotherapy;
providing a second sample obtained from the subject who has received, or who is receiving, allergen immunotherapy;
wherein the first and second samples comprise allergen specific Bmem; and
determining the proportion or number of IL4Rα+ allergen specific Bmem or IL4Rαhi allergen specific Bmem in the first and second samples;
wherein an increase in the proportion or number of IL4Rα+ allergen specific Bmem or IL4Rαhi allergen specific Bmem in the second sample compared to the first sample indicates efficacy of an allergen immunotherapy in a subject.

4. The method according to claim 1, the method further comprising:
determining the proportion or number of CD23+ allergen specific Bmem or CD23hi allergen specific Bmem in the first and second samples;
wherein an increase in the proportion or number of CD23+ and CD29+ allergen specific Bmem or CD23hi and CD29hi allergen specific Bmem in the second sample compared to the first sample indicates efficacy of an allergen immunotherapy in a subject.

5. The method according to claim 1, the method further comprising:
determining the proportion or number of IL4Rα+ allergen specific Bmem or IL4Rαhi allergen specific Bmem in the first and second samples;
wherein an increase in the proportion or number of CD29+ and IL4Rα+ allergen specific Bmem or CD29hi and IL4Rαhi allergen specific Bmem in the second sample compared to the first sample indicates efficacy of an allergen immunotherapy in a subject.

6. The method according to claim 2, the method further comprising:
determining the proportion or number of IL4Rα+ allergen specific Bmem or IL4Rαhi allergen specific Bmem in the first and second samples;
wherein an increase in the proportion or number of CD23+ and IL4Rα+ allergen specific Bmem or CD23hi and IL4Rαhi allergen specific Bmem in the second sample compared to the first sample indicates efficacy of an allergen immunotherapy in a subject.

7. A method of determining efficacy of an allergen immunotherapy in a subject, the method comprising:
providing a first sample obtained from a subject before receiving allergen immunotherapy;
providing a second sample obtained from the subject who has received, or who is receiving, allergen immunotherapy;
wherein the first and second samples comprise allergen specific Bmem; and
determining the proportion or number of CD23+, CD29+ and IL4Rα+ allergen specific Bmem or CD23hi, CD29hi and IL4Rαhi allergen specific Bmem in the first and second samples;
wherein an increase in the proportion or number of CD23+, CD29+ and IL4Rα+ allergen specific Bmem CD23hi, CD29hi and IL4Rαhi allergen specific Bmem in the second sample compared to the first sample indicates efficacy of an allergen immunotherapy in a subject.

8. A method of determining efficacy of an allergen immunotherapy in a subject, the method comprising:
providing a first sample obtained from a subject before receiving allergen immunotherapy;
providing a second sample obtained from the subject who has received, or who is receiving, allergen immunotherapy;
wherein the first and second samples comprise allergen specific Bmem; and
determining the level or amount of CD29 biomarker in the first and second samples;
wherein an increase in the level or amount of CD29 biomarker in the second sample compared to the first sample indicates efficacy of an allergen immunotherapy in a subject.

9. A method of determining efficacy of an allergen immunotherapy in a subject, the method comprising:
providing a first sample obtained from a subject before receiving allergen immunotherapy;
providing a second sample obtained from the subject who has received, or who is receiving, allergen immunotherapy;
wherein the first and second samples comprise allergen specific Bmem; and
determining the level or amount of CD23 biomarker in the first and second samples;
wherein an increase in the level or amount of CD23 biomarker in the second sample compared to the first sample indicates efficacy of an allergen immunotherapy in a subject.

10. A method of determining efficacy of an allergen immunotherapy in a subject, the method comprising:
providing a first sample obtained from a subject before receiving allergen immunotherapy;
providing a second sample obtained from the subject who has received, or who is receiving, allergen immunotherapy;
wherein the first and second samples comprise allergen specific Bmem; and
determining the level or amount of IL4Rα biomarker in the first and second samples;
wherein an increase in the level or amount of IL4Rα biomarker in the second sample compared to the first sample indicates efficacy of an allergen immunotherapy in a subject.

11. The method according to claim 8, the method further comprising:
determining the level or amount of CD23 biomarker in the first and second samples;
wherein an increase in the level or amount of CD29 and CD23 biomarkers in the second sample compared to the first sample indicates efficacy of an allergen immunotherapy in a subject, or
wherein an increase in the level or amount of CD29 biomarker and a decrease in the level or amount of CD23 biomarker in the second sample compared to the first sample indicates efficacy of an allergen immunotherapy in a subject.

12. The method according to claim 8, the method further comprising:
determining the level or amount of IL4Rα biomarker in the first and second samples;
wherein an increase in the level or amount of CD29 and IL4Rα biomarkers in the second sample compared to the first sample indicates efficacy of an allergen immunotherapy in a subject, or
wherein an increase in the level or amount of CD29 biomarker and a decrease in the level or amount of IL4Rα biomarker in the second sample compared to the first sample indicates efficacy of an allergen immunotherapy in a subject.

13. The method according to claim 9, the method further comprising:
determining the level or amount of IL4Rα biomarker in the first and second samples;
wherein an increase in the proportion or number of CD23 and IL4Rα biomarkers in the second sample compared to the first sample indicates efficacy of an allergen immunotherapy in a subject.

14. A method of determining efficacy of an allergen immunotherapy in a subject, the method comprising:
providing a first sample obtained from a subject before receiving allergen immunotherapy;
providing a second sample obtained from the subject who has received, or who is receiving, allergen immunotherapy;
wherein the first and second samples comprise allergen specific Bmem; and
determining the level or amount of CD29, CD23 and IL4Rα biomarkers in the first and second samples;
wherein an increase in the level or amount of CD29, CD23 and IL4Rα biomarkers in the second sample compared to the first sample indicates efficacy of an allergen immunotherapy in a subject, or
wherein an increase in the level or amount of CD29 biomarker and a decrease in CD23 and IL4Rα biomarkers in the second sample compared to the first sample indicates efficacy of an allergen immunotherapy in a subject.

15. The method according to any one of claims 8, 11 to 12 and 14, wherein there is at least a 1.5-fold increase in the level or amount of CD29 biomarker in the second sample compared to the first sample indicating efficacy of an allergen immunotherapy in a subject.

16. The method according to any one of claims 8, 11 to 12 and 14, wherein there is at least a 4-fold increase in the level or amount of CD29 biomarker in the second sample compared to the first sample indicating efficacy of an allergen immunotherapy in a subject.

17. The method according to any one of claims 9 and 12 to 16, wherein there is at least a 1.5-fold increase or decrease in the level or amount of CD23 biomarker in the second sample compared to the first sample indicating efficacy of an allergen immunotherapy in a subject.

18. The method according to any one of claims 9 and 12 to 16, wherein there is at least a 4-fold increase or decrease in the level or amount of CD23 biomarker in the second sample compared to the first sample indicating efficacy of an allergen immunotherapy in a subject.

19. The method according to any one of claims 10 and 12 to 18, wherein there is at least a 1.5-fold increase or decrease in the level or amount of IL4Rα biomarker in the second sample compared to the first sample indicating efficacy of an allergen immunotherapy in a subject.

20. The method according to any one of claims 10 and 12 to 18, wherein there is at least a 4-fold increase or decrease in the level or amount of IL4Rα biomarker in the second sample compared to the first sample indicating efficacy of an allergen immunotherapy in a subject.

21. The method of any one of claims 1 to 20, wherein the allergen specific Bmem are IgG₄+ or IgG₄hi allergen specific Bmem.

22. The method according to any one of claims 8 to 21, the method further comprising:
determining the level or amount of one or more biomarkers in the first and second samples, wherein the biomarkers are selected from the group consisting of IgE, CD69, IL13Rα, CD99, IgD, CXCR4, FCRL3, FCRL2, FCRL5, SIGLEC10, and CD1c;
wherein an increase in the level or amount of one or more biomarkers, selected from IgE, IL13Rα, CD99, FCRL3, FCRL2, SIGLEC10, and CD1c in allergen specific Bmem in the second sample compared to the first sample indicates efficacy of an allergen immunotherapy in a subject, and/or
wherein a decrease in the level or amount of one or more biomarkers, selected from CD69, IgD, CXCR4, FCRL3, FCRL2, FCRL5, and CD1c in allergen specific Bmem in the second sample compared to the first sample indicates efficacy of an allergen immunotherapy in a subject.

23. The method according to claim 22, wherein there is at least a 1.5 fold increase in the level or amount of one or more biomarkers selected from IgE, IL13Rα, CD99, SIGLEC 10 and CD1c in allergen specific Bmem and/or there is at least a 1.5 fold decrease in the level or amount of one or more biomarkers selected from CD69, IgD, CXCR4, FCRL3, FCRL2, FCRL5, and CD1c in allergen specific Bmem in the second sample compared to the first sample indicating efficacy of an allergen immunotherapy in a subject.

24. The method according to claim 22 or 23, wherein there is at least a 1.5 fold increase in the level or amount of one or more biomarkers selected from IgE, CD29, IL13Rα, CD99, SIGLEC10, CD1c and CD23; and
at least a 1.5 fold decrease in the level or amount of one or more biomarkers selected from, CD69, IgD, CXCR4, FCRL3, FCRL2, FCRL5, and CD1c;
in allergen specific Bmem in the second sample compared to the first sample indicating efficacy of an allergen immunotherapy in a subject.

25. The method according to any one of claims 22 to 24, wherein there is at least a 3-fold increase in the level or amount of one or more biomarkers selected from IgE, and IL13Rα;
and at least a 1.5 fold decrease in the level or amount of one or more biomarkers selected from CD69, IgD, CXCR4, FCRL3, FCRL2, FCRL5, and CD1c;
in allergen specific Bmem in the second sample compared to the first sample indicating efficacy of an allergen immunotherapy in a subject.

26. The method according to any one of claims 22 to 25, wherein there is at least a 4-fold increase in the level or amount of CD29;
and at least a 1.5 fold decrease in the level or amount of one or more biomarkers selected from CD69, IgD, CXCR4, FCRL3, FCRL2, FCRL5, and CD1c;
in allergen specific Bmem in the second sample compared to the first sample indicating efficacy of an allergen immunotherapy in a subject.

27. The method according to any one of claims 1 to 26, wherein the second sample is obtained from a subject after the initiation phase of the allergen immunotherapy.

28. The method according to claim 27, wherein the initiation phase of the allergen immunotherapy is at least 4 months.

29. The method according to claim 27, wherein the initiation phase of the allergen immunotherapy is at least 63 days.

30. The method according to any one of claims 1 to 26, wherein the second sample is obtained from the subject at least 4 months after initiation of treatment of the allergen immunotherapy.

31. The method according to any one of claims 1 to 26, wherein the second sample is obtained from the subject between 6 to 12 months after initiation of treatment of the allergen immunotherapy.

32. The method according to any one of claims 1 to 26, wherein the second sample is obtained from the subject up to 5 years after initiation of treatment of the allergen immunotherapy.

33. The method according to any one of claims 1 to 26, wherein the allergen immunotherapy is administered sublingually or subcutaneously.

34. The method according to any of claims 1 to 33, wherein the sample is a whole blood sample.

35. The method according to claim 34, wherein the sample comprises peripheral blood mononuclear cells.

36. The method according to any one of claim 35, wherein the sample is selected for using flow cytometry.

37. The method according to any one of claims 1 to 33, wherein the sample is a serum.

38. The method according to any one of claims 1 to 37, the method further comprising
determining the level or amount of one or more biomarkers in allergen specific Bmem in the first and second samples, wherein the biomarkers are selected from the group consisting of IGHE, PPP1R18, ITGB1, PARM1, c-jun, CD69, FCER2, and IL4R;
wherein an increase in the level or amount of one or more biomarkers selected from IGHE, PPP1R18, ITGB1, PARM1, c-jun, CD69, FCER2, and IL4R in allergen specific Bmem in the second sample compared to the first sample indicates efficacy of an allergen immunotherapy in a subject, and/or
wherein a decrease in the level or amount of one or more biomarkers selected from FCER2, IL4R, c-jun and CD69 in Bmem in the second sample compared to the first sample indicates efficacy of an allergen immunotherapy in a subject.

39. A method of determining efficacy of an allergen immunotherapy in a subject, the method comprising:
providing a first sample obtained from a subject before receiving allergen immunotherapy;
providing a second sample obtained from the subject who has received, or who is receiving, allergen immunotherapy;
wherein the first and second samples comprise allergen specific Bmem; and
determining the level or amount of one or more of FCER2, ITGB1 and IL4R biomarkers in the first and second samples;
wherein an increase in the level or amount of one or more of FCER2, ITGB1 and IL4R biomarkers in the second sample compared to the first sample indicates efficacy of an allergen immunotherapy in a subject, and/or
wherein a decrease in the level or amount of one or more of FCER2 and IL4R biomarkers in the second sample compared to the first sample indicates efficacy of an allergen immunotherapy in a subject.

40. The method according to claim 38 or 39, wherein the level or amount of one or more biomarkers is the level or amount of RNA.

41. The method according to claim 40, wherein the RNA is any one of pre-mRNA or mature mRNA.

42. The method according to any one of claims 1 to 41, wherein the allergen immunotherapy is for treatment of sensitivity to an allergen, wherein the allergen is a recombinant allergen.

43. The method according to any one of claims 1 to 41, wherein the allergen immunotherapy is for treatment of sensitivity to an allergen, wherein the allergen is a synthetic allergen.

44. The method according to any one of claims 1 to 41, wherein the allergen immunotherapy is for treatment of sensitivity to an allergen, wherein the allergen is an environmental allergen.

45. The method according to claim 44, wherein the allergen is a grass pollen allergen.

46. The method according to claim 44, wherein the allergen is a ryegrass pollen allergen.

47. A kit for determining efficacy of an allergen immunotherapy in a subject comprising:
(i) a means of detecting changes in the level or amount of transcripts and/or proteins, wherein the transcripts and/or proteins are selected from the group consisting of ITGB1, IL4R and FCER2 or proteins: CD29, IL4Rα and CD23, or any combination thereof; and
(ii) optionally written instructions for using the kit in a method of any one of claims 1 to 40.

48. A biomarker panel for determining efficacy of allergen immunotherapy of a subject, the panel comprising at least one of the following biomarkers: ITGB1, IL4R and FCER2 or proteins: CD29, IL4Rα and CD23 or any combinations thereof;
wherein measurement or detection of the at least one biomarker indicates that the subject has responded to allergen immunotherapy.

49. A method of determining efficacy of an allergen immunotherapy in a subject, the method comprising:
providing a test sample obtained from a subject who has received, or who is receiving, allergen immunotherapy, wherein the test sample comprises allergen specific Bmem;
determining the proportion or number of one or more of CD29+, CD23+ and IL4Rα+ allergen specific Bmem or CD29hi, CD23hi and IL4Rhi allergen specific Bmem in the test sample; and
comparing the proportion or number of one or more of CD29+, CD23+ and IL4Rα+ allergen specific Bmem or CD29hi, CD23hi and IL4Rαhi allergen specific Bmem in the test sample to reference values, or reference ranges, or a reference standard of CD29+, CD23+ and IL4Rα+ allergen specific Bmem or CD29hi, CD23hi and IL4Rαhi allergen specific Bmem for subjects who have the same or related allergy and who have not received an allergen immunotherapy,
wherein when the proportion or number of one or more of CD29+, CD23+ and IL4Rα+ allergen specific Bmem or CD29hi, CD23hi and IL4Rαhi allergen specific Bmem in the test sample is the same or similar to the reference values, or reference ranges, or a reference standard a determination is made that the subject is not responding to the allergen immunotherapy,
or
wherein when the proportion or number of CD29+, CD23+ and IL4Rα+ allergen specific Bmem or CD29hi, CD23hi and IL4Rαhi allergen specific Bmem in the test sample is different to the reference values, or reference ranges, or a reference standard a determination is made that the subject is responding to the allergen immunotherapy.

50. The method according to claim 49, the method further comprising:
determining the proportion or number of one or more biomarkers in allergen specific Bmem in the test sample, wherein the biomarkers are selected from the group consisting of IgE, CD69, IL13Rα, CD99, IgD, CXCR4, FCRL3, FCRL2, FCRL5, SIGLEC10, and CD1c;
comparing the proportion or number of the one or more biomarkers in allergen specific Bmem in the test sample to reference values, or reference ranges, or a reference standard of the one or more biomarkers for subjects who have the same or related allergy and who have received an allergen immunotherapy,
wherein when the proportion or number of one or more biomarkers in allergen specific Bmem in the test sample is the same or similar to the reference values, or reference ranges, or a reference standard a determination is made that the subject is responding to the allergen immunotherapy, or
wherein when the proportion or number of one or more biomarkers in allergen specific Bmem in the test sample is different to the reference values, or reference ranges, or a reference standard a determination is made that the subject is not responding to the allergen immunotherapy.

51. The method according to claims 49 to 50, wherein the test sample is obtained after the initiation phase of the allergen immunotherapy.

52. The method according to any one of claims 49 to 51, wherein the allergen specific Bmem are IgG₄+ or IgG₄hi allergen specific Bmem.

53. The method according to claims 49 to 52, wherein the allergen specific Bmem is specific for the allergen included in the allergen immunotherapy that the subject has received or is receiving.

54. A kit for determining efficacy of an allergen immunotherapy in a subject comprising:
(i) a means of detecting changes in the level or amount of transcripts and/or proteins, wherein the transcripts and/or proteins are selected from the group consisting of IGHE, ITGB1, CD69, IL13RA1, CD99, IGHD, CSCR3, FCRL3, FCRL2, FCRL5, SIGLEC10, CD1C, IL4R and FCER2 or proteins: IgE, CD29, CD69, IL13Rα, CD99, IgD, CXCR4, FCRL3, FCRL2, FCRL5, SIGLEC10, CD1c, CD23, and IL4Rα, or any combination thereof; and
(ii) optionally written instructions for using the kit in a method of any one of claims 1 to 40.

55. A biomarker panel for determining efficacy of allergen immunotherapy of a subject, the panel comprising at least one of the following biomarkers: IGHE, ITGB1, CD69, IL13RA1, CD99, IGHD, CSCR3, FCRL3, FCRL2, FCRL5, SIGLEC10, CD1C, IL4R and FCER2 or proteins: IgE, CD29, CD69, IL13Rα, CD99, IgD, CXCR4, FCRL3, FCRL2, FCRL5, SIGLEC10, CD1c, CD23, and IL4Rα, or any combinations thereof;
wherein measurement or detection of the at least one biomarker indicates that the subject has responded to allergen immunotherapy.
